(19) United States
(12) Patent Application Publication
(10) Pub. No.: US 2002/0152494 A1
(43) Pub. Date: Oct. 17, 2002

(54) PRODUCTION OF RECOMBINANT ORGANISMS

(76) Inventors: ROY GEOFFREY SARGENT, MOUNTAIN VIEW, CA (US); ANNE KATHRYN VALLERGA, MENLO PARK, CA (US); SUSHMA PATI, LOS ALTOS, CA (US); DAVID ALAN ZARLING, MENLO PARK, CA (US)

Correspondence Address: FLEHR HOHBACH TEST ALBRITTON, & HERBERT LLP, FOUR EMBARCADERO CENTER SUITE 3400, SAN FRANCISCO, CA, 941114187

(21) Appl. No.: 09/470,859

(22) Filed: Dec. 23, 1999

Related U.S. Application Data
(60) Provisional application No. 60/153,795, filed on Sep. 14, 1999.

(51) Int. Cl. A01K 67/027; A01N 1/02; C12N 15/00

(52) U.S. Cl. 800/24; 435/1.1; 600/33

(57) ABSTRACT

A method comprising: a) altering a chromosomal sequence of a donor nucleus of a donor cell by introducing a pair of single-stranded targeting polynucleotides, and a recombinase into said donor nucleus of said donor cell, wherein said pair of targeting polynucleotides are substantially complementary to each other and each comprising a homology clamp that substantially corresponds to or is substantially complementary to a predetermined DNA sequence of said nucleus; and, b) transplanting said nucleus into an oocyte to produce a recombinant zygote. A method of altering a nucleic acid sequence of a mitochondria or chloroplast of a cell comprising: introducing into a cell a pair of single-stranded targeting polynucleotides, and recombinase, wherein said pair of targeting polynucleotides are substantially complementary to each other, and each comprising a homology clamp that substantially corresponds to or is substantially complementary to a predetermined nucleic acid sequence of said mitochondria or chloroplast, whereby said sequence is altered.

This is a continuation-in-part of provisional application serial No.60/153,795, filed Sep. 14, 1999, pending.

### FIELD OF THE INVENTION

The invention relates to compositions and methods of producing recombinant organisms by enhanced homologous recombination.

### BACKGROUND OF THE INVENTION

The cloning of mammals, and other organisms, using nuclear transfer technologies entails removal of the nucleus from an unfertilized female egg or oocyte and implantation of a nucleus, from a donor cell usually of the same species, into the enucleated recipient oocyte. The reconstructed cell or recombinant zygote is activated to induce cell division and the developing embryo is implanted into a surrogate mother. Since the offspring born to these surrogate mothers are genetically identical to the donor cell nuclei used for nuclear transfer, it is possible to generate herds of animals or plant crops with genetically identical individuals, that are genetically identical to the organism from which donor cells were isolated. If genetically modified donor cells are used for nuclear transfer, the resulting offspring will also contain the genetic modification.

The cloning of mammals using nuclei from intact embryonic cells by nuclear transfer has been reported for sheep, cows, goats, mice, rhesus monkeys, pigs, and rabbits. Recently, the cloning of sheep, cows, goats, and mice by nuclear transfer using intact fully differentiated adult cells has also been demonstrated. Genetically engineered cattle, sheep and goats have been cloned by nuclear transfer from intact fetal cells containing randomly integrated transgenes, proving that for these species donor nuclei are competent to support embryonic development after short term growth in cell culture with selective agents. However, genetically engineered clonally derived animals containing gene modifications introduced by homologous recombination at defined chromosomal sites have not been described. This could be due to several factors, however, one likely factor contributing to the quality of nuclei for nuclear transfer is the prolonged growth of nuclei-donor cells in tissue culture leading to genetic or physiological changes that diminish the ability of transferred nuclei to support embryonic development to birth.

Cell lines derived from differentiated tissues that are used for nuclear transfer, however, have limited lifespans in culture. Since engineering genetic modifications in cells by conventional methods requires drug selections and prolonged outgrowth of recombinant cells in culture, cell lines that have limited lifespans in culture currently are not good candidates to be used for production of recombinant organisms by nuclear transfer.

There is a need for methods and compositions designed to introduce genetic modifications in a high frequency of isolated nuclei or nuclei of differentiated cells. High frequency gene modifications using enhanced homologous recombination (EHR) in isolated nuclei or cell populations avoids the need to select for recombinant cells by drug selections and decreases the amount of time cells need to be kept in culture. Since EHR results in gene modifications in several percent of the cells, homologous recombinant cells can be identified by directly screening individual colonies by PCR or Southern hybridization. This high throughput and rapid turnaround in identifying homologous recombinant cells ultimately results in a better quality of recombinant nuclei that can be used to regenerate clonally derived organisms by nuclear transfer.

Another approach to the production of recombinant organisms is intracytoplasmic sperm injection (ISI). In this method spermatozoa are injected into oocytes. Co-injection of exogenous DNA results in integration of the exogenous DNA into the chromosome of the injected cell. Transgenic organisms produced by this method express the exogenous DNA sequences, however the relative number of transgenic organism is low, due to the inefficiency of the integration process by conventional homologous recombination (Perry et al. Science. 284:1180 (1999).

The low efficiency of conventional homologous recombination (CHR) in living cells is dependent on several parameters, including the method of DNA delivery, how it is packaged, its size and conformation, DNA length and position of sequences homologous to the target, and the efficiency of hybridization and recombination at chromosomal sites. These variables severely limit the use of CHR approaches to transgenic organism production. (Kucherlapati et al., 1984. PNAS USA 81:3153-3157; Smithies et al. 1985. Nature 317:230-234; Song et al. 1987. PNAS USA 84:6820-6824; Doetschman et al. 1987. Nature 330:576-578; Kim and Smithies. 1988. Nuc. Acids. Res. 16:8887-8903; Koller and Smithies. 1989. PNAS USA 86:8932-8935; Shesely et al. 1991. PNAS USA 88:4294-4298; Kim et al. 1991. Gene 103:227-233).

The homologous recombination frequency is significantly enhanced by the presence of recombinase activities in cellular and cell free systems. Several proteins or purified extracts that promote homologous recombination (i.e., recombinase activity) have been identified in prokaryotes and eukaryotes (Cox and Lehman., 1987. Annu. Rev. Biochem. 56:229-262; Radding. 1982. Annual Review of Genetics 16:405-547; McCarthy et al. 1988. PNAS USA 85:5854-5858). These recombinases promote one or more steps in the formation of homologously-paired intermediates, strand-exchange, and/or other steps. The most studied recombinase to date is the RecA recombinase of E coli, which is involved in homology search and strand exchange reactions (Cox and Lehman, 1987, supra).

The bacterial RecA protein (Mr 37,842) catalyses homologous pairing and strand exchange between two homologous DNA molecules (Kowalczykowski et al. 1994. Microbiol. Rev. 58:401-465; West. 1992. Annu. Rev. Biochem. 61:603-640); Roca and Cox. 1990. CRC Cit. Rev. Biochem. Mol. Biol. 25:415-455; Radding. 1989. Biochim. Biophys. Acta. 1008:131-145; Smith. 1989. Cell 58:807-809). RecA protein binds cooperatively to any given sequence of single-stranded DNA with a stoichiometry of one RecA protein monomer for every three to four nucleotides in DNA (Cox and Lehman, 1987, supra). This forms unique right handed helical nucleoprotein filaments in which the DNA is extended by 1.5 times its usual length (Yu and Egelman 1992. J. Mol. Biol. 227:334-346). These nucleoprotein filaments, which are referred to as DNA probes, are crucial “homology search engines” which catalyze DNA pairing. Once the filament finds its homologous target gene sequence, the DNA probe strand invades the target and forms a hybrid DNA structure, referred to as a joint molecule or D-loop (DNA displacement loop) (McEntee et al. 1979. PNAS USA 76:2615-2619; Shibata et al. 1979. PNAS USA 76:1638-1642). The phosphate backbone of DNA inside the RecA nucleoprotein filaments is protected against digestion by phosphodiesterases and nucleases.

RecA protein is the prototype of a universal class of recombinase enzymes which promote probe-target pairing reactions. Recently, genes homologous to E. coli RecA (the Rad51 family of proteins) were isolated from all groups of eukaryotes, including yeast and humans. Rad51 protein promotes homologous pairing and strand invasion and exchange between homologous DNA molecules in a similar manner to RecA protein (Sung. 1994. Science 265:1241-1243; Sung and Robberson. 1995. Cell 82:453-461; Gupta et al. 1997. PNAS USA 94:463-468; Baumann et al. 1996. Cell 87:757-766).

Methods and compositions describing enhanced homologous recombination are found in U.S. Pat. No. 5,763,240, WO/93/22443; WO91/17424; and WO98/42727.

Accordingly, an object of the invention to apply methods and compositions of EHR in the production of genetically modified, recombinant or transgenic organisms.

### SUMMARY OF THE INVENTION

The present invention provides methods of altering a chromosomal sequence in a cell to produce a transgenic organism.

In one aspect, the method comprises altering a chromosomal sequence of a donor nucleus by introducing a pair of single-stranded targeting polynucleotides and a recombinase into a nucleus of a cell. The targeting polynucleotides are substantially complementary to each other and each comprises a homology clamp that substantially corresponds to or is substantially complementary to a predetermined sequence of the target chromosomal sequence. The method further comprises transplanting the nucleus into an oocyte to produce a recombinant zygote. The zygote is activated and transferred to a surrogate mother whereby transgenic offspring are produced.

In another aspect, the method comprises altering a chromosomal sequence by introducing a spermatozoa, a pair of single-stranded targeting polynucleotides and a recombinase into an oocyte to produce a recombinant zygote. The targeting polynucleotides are substantially complementary to each other and each comprises a homology clamp that substantially corresponds to or is substantially complementary to a predetermined chromosomal sequence of the spermatozoa and/or oocyte. The recombinant zygote is activated to divide and transferred to a surrogate mother whereby transgenic offspring are produced.

In yet another aspect of the invention, methods and compositions are provided for targeting and altering an extrachromosomal sequence of a cell, such as, a mitochondrial or chloroplast nucleic acid sequence. The method comprises introducing a pair of single-stranded targeting polynucleotides and a recombinase into a cell. The targeting polynucleotides are substantially complementary to each other and each comprises a homology clamp that substantially corresponds to or is substantially complementary to a predetermined sequence of the target extrachromosomal sequence.

In a further aspect the invention provides, the transgenic offspring which are fertile and are inbred or outbread to produce a population of transgenic organisms.

### DETAILED DESCRIPTION OF THE FIGURES

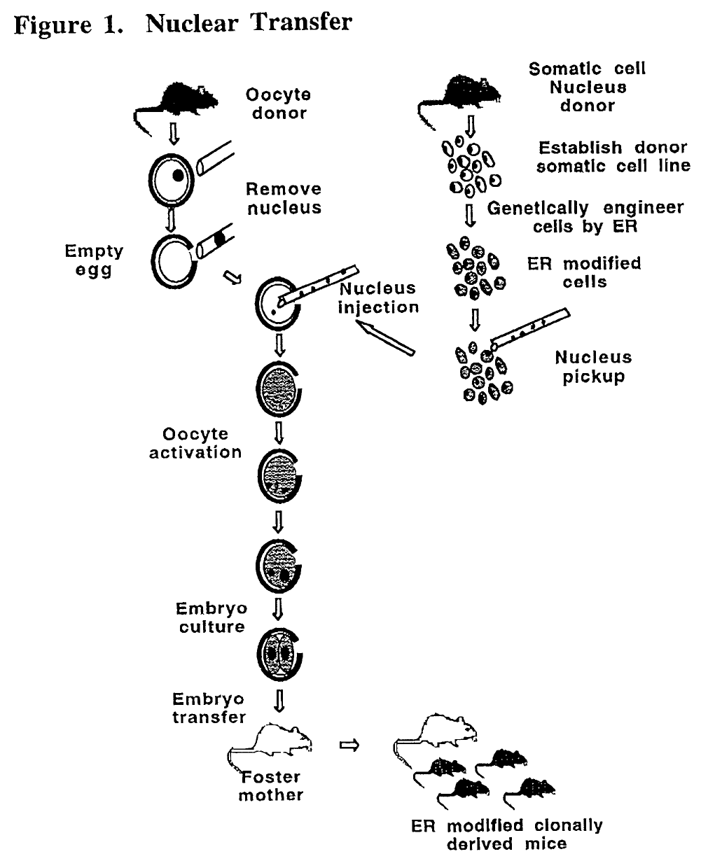

FIG. 1 depicts a method of making enhanced homologous recombination modified clonally derived mice.

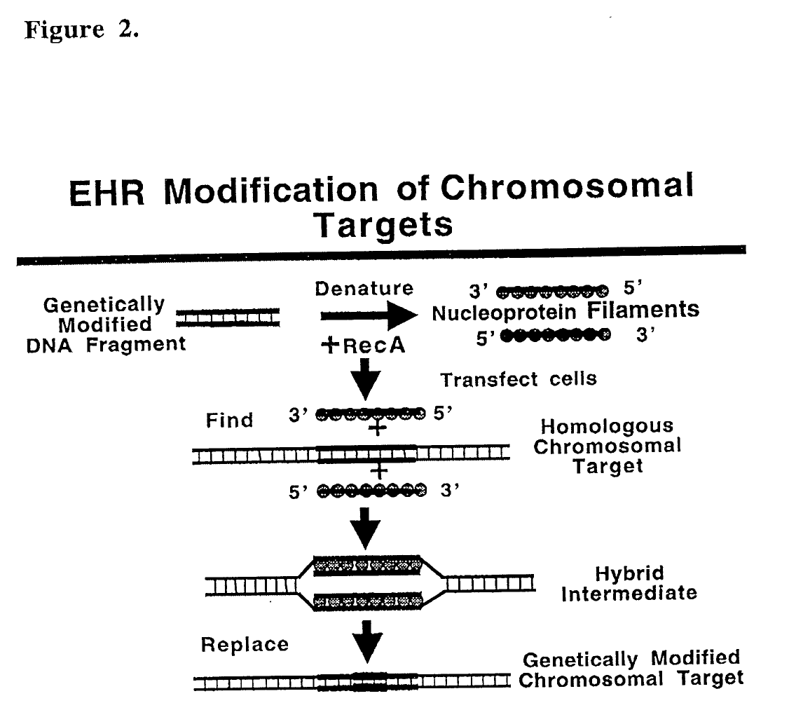

FIG. 2 depicts enhanced homologous recombination modification of chromosomal targets.

### DESCRIPTION OF PREFERRED EMBODIMENTS

The present invention provides methods and compositions for producing a recombinant organism. In one aspect of the invention, the method comprises introducing a pair of single-stranded targeting polynucleotides and a recombinase into a nucleus of a cell. The targeting polynucleotides are substantially complementary to each other and comprise a homology clamp that substantially corresponds to a predetermined DNA sequence of the cell. The targeting polynucleotides and the predetermined DNA sequence undergo enhanced homologous recombination (EHR), thereby modifying the predetermined DNA sequence of the cell. The nucleus is introduced into an enucleated oocyte to produce a recombinant zygote which is activated to divide and transferred into a surrogate mother. In the surrogate mother, the activated zygote develops into a recombinant organism having the targeted DNA sequence modification. The recombinant organisms are harvested and preferably bred to produce a population of recombinant organisms.

In another aspect of the invention, the method comprises introducing a pair of single-stranded targeting polynucleotides, a recombinase, and a spermatozoa into an oocyte. The targeting polynucleotides are substantially complementary to each other and comprise a homology clamp that substantially corresponds to a predetermined DNA sequence of the spermatozoa and/or oocytes. The targeting polynucleotides and the predetermined DNA sequence undergo enhanced homologous recombination to modify the predetermined DNA sequence. The injected oocyte becomes a recombinant zygote which is activated to divide and transferred into a surrogate mother. In the surrogate mother, the activated zygote develops into a recombinant organism having the targeted DNA sequence modificatioin. The recombinant organisms are harvested and preferably bred to produce a population of recombinant organisms.

In yet another aspect of the invention, methods and compositions are provided for targeting and altering a predetermined extrachromosomal sequence of a cell, such as, a mitochondrial or chloroplast nucleic acid sequence. The method comprises introducing a pair of single-stranded targeting polynucleotides and a recombinase into a cell. The targeting polynucleotides are substantially complementary to each other and each comprises a homology clamp that substantially corresponds to or is substantially complementary to a predetermined sequence of the target extrachromosomal sequence. The targeting polynucleotides and the predetermined extrachromosomal sequence undergo enhanced homologous recombination, thereby modifying the predetermined extrachromosomal sequence of the cell.

Accordingly, the methods comprise providing a cell with one or more pairs of single-stranded targeting polynucleotides, a predetermined target nucleic acid, and a recombinase to form a polynucleotide:target nucleic acid complex. The targeting polynucleotides comprise at least one homology clamp for targeting a predetermined DNA sequence and a sequence for modifying at least one nucleotide of the predetermined DNA sequence. Strand exchange and homologous recombination between the targeting polynucleotides and the predetermined DNA sequence modifies the DNA sequence. As described herein, a recombinant zygote comprising the modified predetermined DNA sequence is produced, activated, and transferred into a surrogate mother, resulting in the production of a recombinant organism having the DNA sequence modification. Preferably, the recombinant organisms are inbred or outbread to produce a population of recombinant organisms.

In yet another aspect of the invention, methods and compositions are provided for targeting and altering an extrachromosomal sequence of a cell, such as, a mitochondrial or chloroplast nucleic acid sequence. The method comprises introducing a pair of single-stranded targeting polynucleotides and a recombinase into a cell. The targeting polynucleotides are substantially complementary to each other and each comprises a homology clamp that substantially corresponds to or is substantially complementary to a predetermined sequence of the target extrachromosomal sequence.

Thus, in a preferred embodiment, the present invention provides methods comprising altering a chromosomal sequence of a donor nucleus. By “chromosomal sequence” herein is meant a nucleic acid sequence contained on a chromosome of the donor nucleus.

In an alternative embodiment, the present invention provides methods comprising altering an extrachromosomal sequence of a donor nucleus. By “extrachromosomal sequence” herein is meant a nucleic acid sequence that is not contained on a chromosome and preferably includes mitochondrial or chloroplast nucleic acids.

In a preferred embodiment, the nuclei, cells, recombinant zygotes, as described herein are optionally cryopreserved as known in the art at the convenience of the practitioner.

By “nucleic acid”, “oligonucleotide”, and “polynucleotide” or grammatical equivalents herein means at least two nucleotides covalently linked together. A nucleic acid of the present invention will generally contain phosphodiester bonds, although in some cases nucleic acid analogs are included that may have alternate backbones, comprising, for example, phosphoramide (Beaucage et al., Tetrahedron 49(10):1925 (1993) and references therein; Letsinger, J. Org. Chem. 35:3800 (1970); Sprinzl et al., Eur. J. Biochem. 81:579 (1977); Letsinger et al., Nucl. Acids Res. 14:3487 (1986); Sawai et al, Chem. Lett. 805 (1984), Letsinger et al., J. Am. Chem. Soc. 110:4470 (1988); and Pauwels et al., Chemica Scripta 26:141 91986)), phosphorothioate, phosphorodithioate, O-methylphophoroamidite linkages (see Eckstein, Oligonucleotides and Analogues: A Practical Approach, Oxford University Press), and 25 peptide nucleic acid backbones and linkages (see Egholm, J. Am. Chem. Soc. 114:1895 (1992); Meier et al., Chem. Int. Ed. Engl. 31:1008 (1992); Nielsen, Nature, 365:566 (1993); Carlsson et al., Nature 380:207 (1996), all of which are incorporated by reference). These modifications of the ribose-phosphate backbone or bases may be done to facilitate the addition of other moieties such as chemical constituents, including 2′ O-methyl and 5′ modified substituents, as discussed below, or to increase the stability and half-life of such molecules in physiological environments. Nucleic acids, oligonucleotides, or polynucleotides can be synthesized on an Applied BioSystems oligonucleotide synthesizer according to specifications provided by the manufacturer. Modified oligonucleotides and peptide nucleic acids are made as is generally known in the art.

The nucleic acids may be single stranded or double stranded, as specified, or contain portions of both double stranded or single stranded sequence. The nucleic acid may be DNA, both genomic and cDNA, RNA or a hybrid, where the nucleic acid contains any combination of deoxyribo-and ribo-nucleotides, and any combination of bases, including uracil, adenine, thymine, cytosine, guanine, inosine, xathanine and hypoxathanine, etc. Thus, for example, chimeric DNA-RNA molecules may be used such as described in Cole-Strauss et al., Science 273:1386 (1996) and Yoon et al., PNAS USA 93:2071 (1996), both of which are hereby incorporated by reference.

In general, the targeting polynucleotides may comprise any number of structures, as long as the changes do not substantially effect the functional ability of the targeting polynucleotide to result in homologous recombination. For example, recombinase coating of alternate structures should still be able to occur.

The chromosomal sequence and extrachromosomal sequence comprise a predetermined endogenous nucleic acid sequence to be altered. As used herein, the terms “predetermined endogenous nucleic acid sequence”, “predetermined endogenous DNA sequence”, “predetermined target sequence”, and “predetermined DNA sequence” refer to polynucleotide sequences contained in a target cell. Such sequences include, for example, chromosomal sequences (e.g., sequences that encode the open reading frame of an encoded protein or encode homology motif tags (HMTs), structural genes, regulatory sequences including promoters and enhancers, recombinatorial hotspots, repeat sequences, integrated proviral sequences, hairpins, palindromes), episomal or extrachromosomal sequences (e.g., replicable plasmids or viral or parasitic replication intermediates) including chloroplast and mitochondrial nucleic acid and DNA sequences. By “predetermined” or “pre-selected” is meant that the target sequence may be selected at the discretion of the practitioner on the basis of known or predicted sequence information, and is not constrained to specific sites recognized by certain site-specific recombinases (e.g., FLP recombinase or CRE recombinase). In some embodiments, the predetermined endogenous DNA target sequence will be other than a naturally occurring germline DNA sequence (e.g., a transgene, parasitic, mycoplasmal or viral sequence). An exogenous polynucleotide is a polynucleotide which is transferred into a target cell but which has not been replicated in that host cell; for example, a virus genome or polynucleotide that enters a cell by fusion of a virion to the cell is an exogenous polynucleotide, however, replicated copies of the viral polynucleotide subsequently made in the infected cell are endogenous sequences (and may, for example, become integrated into a cell chromosome). Similarly, transgenes which are microinjected or transfected into a cell are exogenous polynucleotides, however integrated and replicated copies of the transgene(s) are endogenous sequences.

In a preferred embodiment, rather than an exact chromosomal sequence being used as the predetermined nucleic acid, a homology motif tag is used. By “homology motif tag” or “protein consensus sequence” herein is meant an amino acid consensus sequence of a gene family. By “consensus nucleic acid sequence” herein is meant a nucleic acid that encodes a consensus protein sequence of a functional domain of a gene family. In addition, “consensus nucleic acid sequence” can also refer to cis sequences that are non-coding but can serve a regulatory or other role. In a preferred embodiment, generally a library of consensus nucleic acid sequences are used, that comprises a set of degenerate nucleic acids encoding the protein consensus sequence. A wide variety of protein consensus sequences for a number of gene families are known. A “gene family” therefore is a set of genes that encode proteins that contain a functional domain for which a consensus sequence can be identified. However, in some instances, a gene family includes non-coding sequences; for example, consensus regulatory regions can be identified. For example, gene family/consensus sequences pairs are known for the G-protein coupled receptor family, the AAA-protein family, the bZIP transcription factor family, the mutS family, the recA family, the Rad51 family, the dmel family, the recF family, the SH2 domain family, the Bcl-2 family, the single-stranded binding protein family, the TFIID transcription family, the TGF-beta family, the TNF family, the XPA family, the XPG family, actin binding proteins, bromodomain GDP exchange factors, MCM family, ser/thr phosphatase family, etc. As will be appreciated by those in the art, the proteins of the gene families generally do not contain the exact consensus sequences; generally consensus sequences are artificial sequences that represent the best comparison of a variety of sequences. The actual sequence that corresponds to the functional sequence within a particular protein is termed a “consensus functional domain” herein; that is, a consensus functional domain is the actual sequence within a protein that corresponds to the consensus sequence. In this way, alterations may be made in any number of gene families. Accordingly, by targeting consensus motifs, targeted modifications may be made in those instances when sequence information is limited.

The term “corresponds to” is used herein to mean that a polynucleotide sequence is homologous (i.e., may be similar or identical, not strictly evolutionarily related) to all or a portion of a reference polynucleotide sequence, or that a polypeptide sequence is identical to a reference polypeptide sequence. In contradistinction, the term “complementary to” is used herein to mean that the complementary sequence is homologous to all or a portion of a reference polynucleotide sequence. As outlined below, preferably, the homology is at least 50-70%, preferably 85%, and more preferably 95% identical. Thus, the complementarity between two single-stranded targeting polynucleotides need not be perfect. For illustration, the nucleotide sequence “TATAC” corresponds to a reference sequence “TATAC” and is perfectly complementary to a reference sequence “GTATA”.

The term “percent (%) nucleic acid sequence identity” is defined as the percentage of nucleotide residues that are identical in the alignment of nucleic acid sequences. A preferred method of determining percent nucleic acid sequence identity utilizes the BLASTN module of WU-BLAST-2 set to the default parameters, with overlap span and overlap fraction set to 1 and 0.125, respectively.

As is known in the art, a number of different programs can be used to identify whether a protein (or nucleic acid as discussed below) has sequence identity or similarity to a known sequence. Sequence identity and/or similarity is determined using standard techniques known in the art, including, but not limited to, the local sequence identity algorithm of Smith & Waterman, Adv. Appl. Math., 2:482 (1981), by the sequence identity alignment algorithm of Needleman & Wunsch, J. Mol. Biol., 48:443 (1970), by the search for similarity method of Pearson & Lipman, Proc. Natl. Acad. Sci. U.S.A., 85:2444 (1988), by computerized implementations of these algorithms (GAP, BESTFIT, FASTA, and TFASTA in the Wisconsin Genetics Software Package, Genetics Computer Group, 575 Science Drive, Madison, Wis.), the Best Fit sequence program described by Devereux et al., Nucl. Acid Res., 12:387-395 (1984), preferably using the default settings, or by inspection. Preferably, percent identity is calculated by FastDB based upon the following parameters: mismatch penalty of 1; gap penalty of 1; gap size penalty of 0.33; and joining penalty of 30, “Current Methods in Sequence Comparison and Analysis,” Macromolecule Sequencing and Synthesis, Selected Methods and Applications, pp 127-149 (1988), Alan R. Liss, Inc, all of which are expressly incorporated by reference.

An example of a useful algorithm is PILEUP. PILEUP creates a multiple sequence alignment from a group of related sequences using progressive, pairwise alignments. It can also plot a tree showing the clustering relationships used to create the alignment. PILEUP uses a simplification of the progressive alignment method of Feng & Doolittle, J. Mol. Evol. 35:351-360 (1987); the method is similar to that described by Higgins & Sharp CABIOS 5:151-153 (1989), both of which are expressly incorporated by reference. Useful PILEUP parameters including a default gap weight of 3.00, a default gap length weight of 0.10, and weighted end gaps.

Another example of a useful algorithm is the BLAST algorithm, described in Altschul et al., J. Mol. Biol., 215, 403-410, (1990) and Karlin et al., Proc. Natl. Acad. Sci. U.S.A., 90:5873-5787 (1993). A particularly useful BLAST program is the WU-BLAST-2 program which was obtained from Altschul et al., Methods in Enzymology, 266:460-480 (1996); http://blast.wustl/edu/blast/ README.html], all of which are expressly incorporated by reference. WU-BLAST-2 uses several search parameters, most of which are set to the default values. The adjustable parameters are set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity.

An additional useful algorithm is gapped BLAST as reported by Altschul et al., Nucl. Acids Res., 25:3389-3402, expressly incorporated by reference. Gapped BLAST uses BLOSUM-62 substitution scores; threshold T parameter set to 9; the two-hit method to trigger ungapped extensions; charges gap lengths of k a cost of 10+k; Xᵤ set to 16, and X_{g} set to 40 for database search stage and to 67 for the output stage of the algorithms. Gapped alignments are triggered by a score corresponding to ˜22 bits.

A % amino acid sequence identity value is determined by the number of matching identical residues divided by the total number of residues of the “longer” sequence in the aligned region. The “longer” sequence is the one having the most actual residues in the aligned region (gaps introduced by WU-Blast-2 to maximize the alignment score are ignored).

In a similar manner, “percent (%) nucleic acid sequence identity” with respect to the coding sequence of the polypeptides identified herein is defined as the percentage of nucleotide residues in a candidate sequence that are identical with the nucleotide residues in the coding sequence of the cell cycle protein. A preferred method utilizes the BLASTN module of WU-BLAST-2 set to the default parameters, with overlap span and overlap fraction set to 1 and 0.125, respectively.

The nucleic acid alignment may include the introduction of gaps in the sequences to be aligned. In addition, for sequences which contain either more or fewer nucleotides than the nucleic acid to which it is being aligned, it is understood that in one embodiment, the percentage of sequence identity will be determined based on the number of identical nucleotides in relation to the total number of nucleotides. Thus, for example, sequence identity is determined using the number of nucleic acids in the shorter sequence, in one embodiment. In percent identity calculations relative weight is not assigned to various manifestations of sequence variation, such as, insertions, deletions, substitutions, etc.

In one embodiment, only identities are scored positively (+1) and all forms of sequence variation including gaps are assigned a value of “0”, which obviates the need for a weighted scale or weighted parameters. Percent sequence identity can be calculated, for example, by dividing the number of matching identical residues by the total number of residues of the “shorter” sequence in the aligned region and multiplying by 100. The “longer” sequence is the one having the most actual residues in the aligned region.

The terms “substantially corresponds to” or “substantial identity” or “homologous” as used herein denotes a characteristic of a nucleic acid sequence, wherein a nucleic acid sequence has at least about 60 percent sequence identity as compared to a reference sequence, typically at least about 75 percent sequence identity, and preferably at least about 95 percent sequence identity as compared to a reference sequence. The percentage of sequence identity is calculated excluding small deletions or additions which total less than 25 percent of the reference sequence. The reference sequence may be a subset of a larger sequence, such as a portion of a gene or flanking sequence, or a repetitive portion of a chromosome. However, the reference sequence is at least 12-18 nucleotides long, typically at least about 30 nucleotides long, and preferably at least about 50 to 100 nucleotides long. “Substantially complementary” as used herein refers to a sequence that is complementary to a sequence that substantially corresponds to a reference sequence. In general, targeting efficiency increases with the length of the targeting polynucleotide portion that is substantially complementary to a reference sequence present in the target DNA.

“Specific hybridization” is defined herein as the formation of hybrids between a targeting polynucleotide (e.g., a polynucleotide of the invention which may include substitutions, deletion, and/or additions as compared to the predetermined target DNA sequence) and a predetermined target DNA, wherein the targeting polynucleotide preferentially hybridizes to the predetermined target DNA such that, for example, at least one discrete band can be identified on a Southern blot of DNA prepared from target cells that contain the target DNA sequence, and/or a targeting polynucleotide in an intact nucleus localized to a discrete chromosomal location characteristic of a unique or repetitive sequence. In some instances, a target sequence may be present in more than one target polynucleotide species (e.g., a particular target sequence may occur in multiple members of a gene family or in a known repetitive sequence, such as, a homology motif tag (HMT)). It is evident that optimal hybridization conditions will vary depending upon the sequence composition and length(s) of the targeting polynucleotide(s) and target(s), and the experimental method selected by the practitioner. Various guidelines may be used to select appropriate hybridization conditions (see, Maniatis et al., Molecular Cloning: A Laboratory Manual (1989), 2nd Ed., Cold Spring Harbor, N.Y. and Berger and Kimmel, Methods in Enzymology, Volume 152, Guide to Molecular Cloning Techniques (1987), Academic Press, Inc., San Diego, Calif., which are incorporated herein by reference.

For example, high stringency conditions are known in the art; see for example Maniatis et al., Molecular Cloning: A Laboratory Manual, 2d Edition, 1989, and Short Protocols in Molecular Biology, ed. Ausubel, et al., both of which are hereby incorporated by reference. Stringent conditions are sequence-dependent and will be different in different circumstances. Longer sequences hybridize specifically at higher temperatures. An extensive guide to the hybridization of nucleic acids is found in Tijssen, Techniques in Biochemistry and Molecular Biology—Hybridization with Nucleic Acid Probes, “Overview of principles of hybridization and the strategy of nucleic acid assays” (1993). Generally, stringent conditions are selected to be about 5-10° C. lower than the thermal melting point (Tₘ) for the specific sequence at a defined ionic strength pH. The Tₘ is the temperature (under defined ionic strength, pH and nucleic acid concentration) at which 50% of the probes complementary to the target hybridize to the target sequence at equilibrium (as the target sequences are present in excess, at Tₘ, 50% of the probes are occupied at equilibrium). Stringent conditions will be those in which the salt concentration is less than about 1.0 M sodium ion concentration, typically about 0.01 to 1.0 M sodium ion concentration (or other salts) at pH 7.0 to 8.3 and the temperature is at least about 30° C. for short probes (e.g. 10 to 50 nucleotides) and at least about 60° C. for long probes (e.g. greater than 50 nucleotides). Stringent conditions may also be achieved with the addition of destabilizing agents such as formamide.

In another embodiment, less stringent hybridization conditions are used; for example, moderate or low stringency conditions may be used, as are known in the art; see Maniatis and Ausubel, supra, and Tijssen, supra.

Methods of hybridizing targeting polynucleotides to a discrete chromosomal location in intact nuclei are provided herein.

In a preferred embodiment, the targeting polynucleotides are directed to a disease allele gene. As used herein, the term “disease allele” refers to an allele of a gene which is capable of producing a recognizable disease. A disease allele may be dominant or recessive and may produce disease directly or when present in combination with a specific genetic background or pre-existing pathological condition. A disease allele also may carry single or multiple mutations and may produce a spectrum of symptoms that vary broadly in severity. For example, a disease allele may render an organism susceptible to a disease. A disease allele may be present in the gene pool or may be generated de novo in an individual by somatic mutation. For example and without limitation, disease alleles include: activated oncogenes, a sickle cell anemia allele, a Tay-Sachs allele, a cystic fibrosis allele, a Lesch-Nyhan allele, a retinoblastoma-susceptibility allele, a Fabry's disease allele, Huntington's chorea allele, and an infectious disease receptor allele. As used herein, a disease allele encompasses both alleles associated with human diseases and alleles associated with recognized veterinary diseases. For example, the AF508 CFTR allele in a human disease allele which is associated with cystic fibrosis in North Americans.

Thus, the present invention provides targeting polynucleotides. By “targeting polynucleotides” herein is meant the polynucleotides used to make alterations in a predetermined target DNA sequence. Targeting polynucleotides may be produced by chemical synthesis of oligonucleotides, nick-translation of a double-stranded DNA template, polymerase chain-reaction amplification of a sequence (or ligase chain reaction amplification), purification of prokaryotic or target cloning vectors harboring a sequence of interest (e.g., a cloned cDNA or genomic clone, or portion thereof) such as plasmids, phagemids, YACs, cosmids, bacteriophage DNA, other viral DNA or replication intermediates, or purified restriction fragments thereof, as well as other sources of single and double-stranded polynucleotides having a desired nucleotide sequence. Targeting polynucleotides are generally ssDNA or dsDNA, most preferably two complementary single-stranded DNAs.

Targeting polynucleotides are generally at least about 2 to 100 nucleotides long, preferably at least about 5 to 100 nucleotides long, at least about 250 to 500 nucleotides long, more preferably at least about 500 to 2000 nucleotides long, or longer; however, as the length of a targeting polynucleotide increases beyond about 20,000 to 50,000 to 400,000 nucleotides, the efficiency of transferring an intact targeting polynucleotide into the cell decreases. The length of homology may be selected at the discretion of the practitioner on the basis of the sequence composition and complexity of the predetermined endogenous target DNA sequence(s) and guidance provided in the art, which generally indicates that 1.3 to 6.8 kilobase segments of homology are preferred (Hasty et al. (1991) Molec. Cell. Biol. 11: 5586; Shulman et al. (1990) Molec. Cell. Biol. 10: 4466, which are incorporated herein by reference). Targeting polynucleotides have at least one sequence that substantially corresponds to, or is substantially complementary to, a predetermined endogenous DNA sequence (i.e., a DNA sequence of a polynucleotide located in a target cell, such as a chromosomal, mitochondrial, chloroplast, viral, episomal, or mycoplasmal polynucleotide). By “substantially complementary” as used herein refers to a sequence that is complementary to a sequence that substantially corresponds to a reference sequence. In general, targeting efficiency increases with the length of the targeting polynucleotide portion that is substantially complementary to a reference sequence present in the predetermined target DNA. Such targeting polynucleotide sequences serve as templates for homologous pairing with the predetermined endogenous sequence(s), and are also referred to herein as homology clamps. In targeting polynucleotides, such homology clamps are typically located at or near the 5′ or 3′ end, preferably homology clamps are internally or located at each end of the polynucleotide (Berinstein et al. (1992) Molec. Cell. Biol. 12: 360, which is incorporated herein by reference). Without wishing to be bound by any particular theory, it is believed that the addition of recombinases permits efficient gene targeting with targeting polynucleotides having short (i.e., about 50 to 1000 basepair long) segments of homology, as well as with targeting polynucleotides having longer segments of homology.

Therefore, it is preferred that targeting polynucleotides of the invention have homology clamps that are highly homologous to the predetermined target endogenous DNA sequence(s), most preferably isogenic. Typically, targeting polynucleotides of the invention have at least one homology clamp that is at least about 18 to 35 nucleotides long, and it is preferable that homology clamps are at least about 20 to 100 nucleotides long, and more preferably at least about 100-500 nucleotides long, although the degree of sequence homology between the homology clamp and the targeted sequence and the base composition of the targeted sequence will determine the optimal and minimal clamp lengths (e.g., G-C rich sequences are typically more thermodynamically stable and will generally require shorter clamp length). Therefore, both homology clamp length and the degree of sequence homology can only be determined with reference to a particular predetermined sequence, but homology clamps generally must be at least about 12 nucleotides long and must also substantially correspond or be substantially complementary to a predetermined target sequence. Preferably, a homology clamp is at least about 12, and preferably at least about 50 nucleotides long and is identical to or complementary to a predetermined target sequence. Without wishing to be bound by a particular theory, it is believed that the addition of recombinases to a targeting polynucleotide enhances the efficiency of homologous recombination between homologous, nonisogenic sequences (e.g., between an exon 2 sequence of a albumin gene of a Balb/c mouse and a homologous albumin gene exon 2 sequence of a C57/BL6 mouse), as well as between isogenic sequences.

The formation of heteroduplex joints or “D-loops” is not a stringent process under certain conditions; genetic evidence supports the view that the classical phenomena of meiotic gene conversion and aberrant meiotic segregation result in part from the inclusion of mismatched base pairs in heteroduplex joints, and the subsequent correction of some of these mismatched base pairs before replication. Observations of recA protein have provided information on parameters that affect the discrimination of relatedness from perfect or near-perfect homology and that affect the inclusion of mismatched base pairs in heteroduplex joints. The ability of recA protein and other recombinases to drive strand exchange past all single base-pair mismatches and to form extensively mismatched joints in superhelical DNA reflect its role in recombination and gene conversion. This error-prone process may also be related to its role in mutagenesis. RecA-mediated pairing reactions involving DNA of φX174 and G4, which are about 70 percent homologous, have yielded homologous recombinants (Cunningham et al. (1981) Cell 24: 213), although recA preferentially forms homologous joints between highly homologous sequences, and is implicated as mediating a homology search process between an invading DNA strand and a recipient DNA strand, producing relatively stable heteroduplexes at regions of high homology. Accordingly, it is the fact that recombinases can drive the homologous recombination reaction between strands which are significantly, but not perfectly, homologous, which allows gene conversion and the modification of target sequences. Thus, targeting polynucleotides may be used to introduce nucleotide substitutions, insertions and deletions into an endogeneous DNA sequence, and thus the corresponding amino acid substitutions, insertions and deletions in proteins expressed from the endogeneous DNA sequence. Methods and compositions that have been used to target and alter, by homologous recombination, substitutions, including insertions and deletions in target sequences have been described; see U.S. application Ser. Nos. 08/381,634; 08/882,756; 09/301,153; 08/781,329; 09/288,586; 09/209,676; 09/007,020; 09/179,916; 09/182,102; 09/182,097; 09/181,027; 09/260,624; and international application nos. US97/19324; US98/26498; US98/01825, all of which are expressly incorporated by reference in their entirety.

In a preferred embodiment, two substantially complementary targeting polynucleotides are used. In one embodiment, the targeting polynucleotides form a double stranded hybrid, which may be coated with recombinase, although when the recombinase is RecA, the loading conditions may be somewhat different from those used for single stranded nucleic acids.

In a prefered embodiment, two substantially complementary single-stranded targeting polynucleotides are used. The two complementary single-stranded targeting polynucleotides are usually of equal length, although this is not required. However, as noted below, the stability of the four strand containing hybrids of the invention is putatively related, in part, to the lack of significant unhybridized single-stranded nucleic acid, and thus significant unpaired sequences are not preferred. Furthermore, as noted above, the complementarity between the two targeting polynucleotides need not be perfect. The two complementary single-stranded targeting polynucleotides are simultaneously or contemporaneously introduced into a target cell harboring a predetermined endogenous target sequence, generally with at least one recombinase protein (e.g., recA). Under most circumstances, it is preferred that the targeting polynucleotides are incubated with recA or other recombinase prior to introduction into a target cell, so that the recombinase protein(s) may be “loaded” onto the targeting polynucleotide(s), to coat the nucleic acid, as is described below, to produce nucleoprotein filaments. Incubation conditions for such recombinase loading are described infra, and also in U.S. Ser. No. 07/755,462, filed Sep. 4, 1991; U.S. Ser. No. 07/910,791, filed Jul. 9, 1992; and U.S. Ser. No. 07/520,321, filed May 7, 1990, each of which is incorporated herein by reference. A targeting polynucleotide may contain a sequence that enhances the loading process of a recombinase, for example a recA loading sequence is the recombinogenic and recombinase nucleation sequence poly[d(A-C)] and its complement, poly[d(G-T)]. The duplex sequence oligo[d(A-C)ₙ•d(G-T)ₙ], where n is from 4 to 35, is a middle repetitive element in target DNA.

There appears to be a fundamental difference in the stability of RecA-protein-mediated D-loops formed between one single-stranded DNA (ssDNA) probe hybridized to negatively supercoiled DNA targets in comparison to relaxed or linear duplex DNA targets. Internally located dsDNA target sequences on relaxed linear DNA targets hybridized by ssDNA probes produce single D-loops, which are unstable after removal of RecA protein (Adzuma, Genes Devel. 6:1679 (1992); Hsieh et al, PNAS USA 89:6492 (1992); Chiu et al., Biochemistry 32:13146 (1993)). This probe DNA instability of hybrids formed with linear duplex DNA targets is most probably due to the incoming ssDNA probe W-C base pairing with the complementary DNA strand of the duplex target and disrupting the base pairing in the other DNA strand. The required high free-energy of maintaining a disrupted DNA strand in an unpaired ssDNA conformation in a protein-free single-D-loop apparently can only be compensated for either by the stored free energy inherent in negatively supercoiled DNA targets or by base pairing initiated at the distal ends of the joint DNA molecule, allowing the exchanged strands to freely intertwine.

However, the addition of a second complementary ssDNA to the three-strand-containing single-D-loop stabilizes the deproteinized hybrid joint molecules by allowing W-C base pairing of the probe with the displaced target DNA strand. The addition of a second RecA-coated complementary ssDNA (cssDNA) strand to the three-strand containing single D-loop stabilizes deproteinized hybrid joints located away from the free ends of the duplex target DNA (Sena & Zarling, Nature Genetics 3:365 (1993); Révet et al. J. Mol. Biol. 232:779 (1993); Jayasena and Johnston, J. Mol. Bio. 230:1015 (1993)). The resulting four-stranded structure, named a double D-loop by analogy with the three-stranded single D-loop hybrid has been shown to be stable in the absence of RecA protein. This stability likely occurs because the restoration of W-C basepairing in the parental duplex would require disruption of two W-C basepairs in the double-D-loop (one W-C pair in each heteroduplex D-loop). Since each base-pairing in the reverse transition (double-D-loop to duplex) is less favorable by the energy of one W-C basepair, the pair of cssDNA probes are thus kinetically trapped in duplex DNA targets in stable hybrid structures. The stability of the double-D loop joint molecule within internally located probe:target hybrids is an intermediate stage prior to the progression of the homologous recombination reaction to the strand exchange phase. The double D-loop permits isolation of stable multistranded DNA recombination intermediates.

In addition, when the targeting polynucleotides are used to generate insertions or deletions in an endogeneous nucleic acid sequence, the use of two complementary single-stranded targeting polynucleotides allows the use of internal homology clamps as depicted in FIG. 13. The use of internal homology clamps allows the formation of stable deproteinized cssDNA:probe target hybrids with homologous DNA sequences containing either relatively small or large insertions and deletions within a homologous DNA target. Without being bound by theory, it appears that these probe:target hybrids, with heterologous inserts in the cssDNA probe, are stabilized by the re-annealing of cssDNA probes to each other within the double-D-loop hybrid, forming a novel DNA structure with an internal homology clamp. Similarly stable double-D-loop hybrids formed at internal sites with heterologous inserts in the linear DNA targets (with respect to the cssDNA probe) are equally stable. Because cssDNA probes are kinetically trapped within the duplex target, the multi-stranded DNA intermediates of homologous DNA pairing are stabilized and strand exchange is facilitated.

In a preferred embodiment, the length of the internal homology clamp (i.e. the length of the insertion or deletion) is from about 1 to 50% of the total length of the targeting polynucleotide, with from about 1 to about 20% being preferred and from about 1 to about 10% being especially preferred, although in some cases the length of the deletion or insertion may be significantly larger. As for the targeting homology clamps, the complementarity within the internal homology clamp need not be perfect.

The invention may also be practiced with individual targeting polynucleotides which do not comprise part of a complementary pair. In each case, a targeting polynucleotide is introduced into a target cell simultaneously or contemporaneously with a recombinase protein, typically in the form of a recombinase coated targeting polynucleotide as outlined herein (i.e., a polynucleotide pre-incubated with recombinase wherein the recombinase is noncovalently bound to the polynucleotide; generally referred to in the art as a nucleoprotein filament).

A targeting polynucleotide used in a method of the invention typically is a single-stranded nucleic acid, usually a DNA strand, or derived by denaturation of a duplex DNA, which is complementary to one (or both) strand(s) of the target duplex nucleic acid. Thus, one of the complementary single stranded targeting polynucleotides is complementary to one strand of the endogeneous target sequence (i.e. Watson) and the other complementary single stranded targeting polynucleotide is complementary to the other strand of the endogeneous target sequence (i.e. Crick). The homology clamp sequence preferably contains at least 90-95% sequence homology with the target sequence, to insure sequence-specific targeting of the targeting polynucleotide to the endogenous DNA target. Each single-stranded targeting polynucleotide is typically about 50-600 bases long, although a shorter or longer polynucleotide may also be employed. Alternatively, targeting polynucleotides may be prepared in single-stranded form by oligonucleotide synthesis methods, which may first require, especially with larger targeting polynucleotides, formation of subfragments of the targeting polynucleotide, typically followed by splicing of the subfragments together, typically by enzymatic ligation.

By “recombinase” herein is meant proteins that, when included with an exogenous targeting polynucleotide, provide a measurable increase in the recombination frequency and/or localization frequency between the targeting polynucleotide and an endogenous predetermined DNA sequence. Thus, in a preferred embodiment, increases in recombination frequency from the normal range of 10⁻⁸ to 10⁻⁴, to 10⁻⁴ to 10¹, preferably 10⁻³ to 10¹, and most preferably 10⁻² to 10¹, may be acheived.

In the present invention, recombinase refers to a family of RecA and RecA-like recombination proteins all having essentially all or most of the same functions, particularly: (i) the recombinase protein's ability to properly bind to and position targeting polynucleotides on their homologous targets and (ii) the ability of recombinase protein/targeting polynucleotide complexes to efficiently find and bind to complementary endogenous sequences. The best characterized recA protein is from E. coi, in addition to the wild-type protein a number of mutant recA-like proteins have been identified (e.g., recA803; see Madiraju et al., PNAS USA 85(18):6592 (1988); Madiraju et al, Biochem. 31:10529 (1992); Lavery et al., J. Biol. Chem. 267:20648(1992)). Further, many organisms have recA-like recombinases with strand-transfer activities. The art teaches several examples of recombinase proteins, for example, from Drosophila, yeast, plant, human, and non-human mammalian cells, including proteinswith biological properties similarto recA (i.e., recA-like recombinases), such as Rad51 from mammals and yeast, and Pk-rec (Rashid et al., Nucleic Acid Res. 25(4):719 (1997)). Accordingly, the RecA family members include but are not limited to E. coli recA, Rec1, Rec2, Rad51 (Sung et al. Science 265 1241 (1994); Baumann et al. Cell 87:757 (1996), Rad51B, Rad51C, Rad51D, Rad51E (Dosangh et al. Nucleic Acids Res. 26:1179-1184 (1998), XRCC2, T4 uvsX, DMC1 (see also Cox and Lehman (1987) Ann. Rev. Biochem. 56: 229; Radding, C. M. (1982) op.cit:: 5854; Lopez et al. (1987) op.cit.; Fugisawa et al., (1985) Nucl. Acids Res. 13: 7473; Hsieh et al., (1986) Cell 44: 885; Hsieh et al., (1989) J. Biol. Chem. 264: 5089; Fishel et al., (1988) Proc. Natl. Acad. Sci. (USA) 85: 3683; Cassuto et al., (1987) Mol. Gen. Genet. 208: 10; Ganea et al., (1987) Mol. Cell Biol. 7: 3124; Moore et al., (1990) J. Biol. Chem. 19: 11108; Keene et al., (1984) Nucl. Acids Res. 12: 3057; Kimeic, (1984) Cold Spring Harbor Svmp. 48: 675; Kmeic, (1986) Cell 44: 545; Kolodner et al., (1987) Proc. Natl. Acad. Sci. USA 84: 5560; Sugino et al., (1985) Proc. Natl. Acad. Sci. USA 85: 3683; Halbrook et al., (1989) J. Biol. Chem. 264: 21403; Eisen et al., (1988) Proc. Natl. Acad. Sci. USA 85: 7481; McCarthy et al., (1988) Proc. Natl. Acad. Sci. USA 85: 5854; Lowenhaupt et al., (1989) J. Biol. Chem. 264: 20568, which are incorporated herein by reference. Further examples of such recombinase proteins include, for example but are not limited to: recA803, uvsX, and other recA mutants and recA-like recombinases (Roca, A. I. (1990) Crit. Rev. Biochem. Molec. Biol. 25: 415), sep1 (Kolodner et al. (1987) Proc. Natl. Acad. Sci. (U.S.A.) 84:5560; Tishkoff et al. Molec. Cell. Biol. 11:2593), RuvC (Dunderdale et al. (1991) Nature 354: 506), DST2, KEM1, XRN1 (Dykstra et al. (1991) Molec. Cell. Biol. 11:2583), STP″/DST1 (Clark et al. (1991) Molec. Cell. Biol. 11:2576), HPP-1 (Moore et al. (1991) Proc. Natl. Acad. Sci. (U.S.A.) 88:9067), other target recombinases (Bishop et al. (1992) Cell 69: 439; Shinohara et al. (1992) Cell 69: 457); incorporated herein by reference. RecA may be purified from E. coli strains, such as E. coli strains JC12772 and JC15369 (available from A. J. Clark and M. Madiraju, University of California-Berkeley, or purchased commercially). These strains contain the recA coding sequences on a “runaway” replicating plasmid vector present at a high copy numbers per cell. The recA803 protein is a high-activity mutant of wild-type recA.

In addition, the recombinase may actually be a complex of proteins, i.e. a “recombinosome”. In addition, included within the definition of a recombinase are portions or fragments of recombinases which retain recombinase biological activity, as well as variants or mutants of wild-type recombinases which retain biological activity, such as the E. coli recA803 mutant with enhanced recombinase activity.

In a preferred embodiment, recA or rad51 is used. For example, recA protein is typically obtained from bacterial strains that overproduce the protein: wild-type E. coli recA protein and mutant recA803 protein may be purified from such strains. Alternatively, recA protein can also be purchased from, for example, Pharmacia (Piscataway, N.J.).

RecA proteins, and its homologs, form a nucleoprotein filament when it coats a single-stranded DNA. In this nucleoprotein filament, one monomer of recA protein is bound to about 3 nucleotides. This property of recA to coat single-stranded DNA is essentially sequence independent, although particular sequences favor initial loading of recA onto a polynucleotide (e.g., nucleation sequences). The nucleoprotein filament(s) can be formed on essentially any DNA molecule and can be formed in cells (e.g., mammalian cells), forming complexes with both single-stranded and double-stranded DNA, although the loading conditions for dsDNA are somewhat different than for ssDNA.

The conditions used to coat targeting polynucleotides with recombinases such as recA protein and ATP_{Y}S have been described in commonly assigned U.S. Ser. No. 07/910,791, filed Jul. 9, 1992; U.S. Ser. No. 07/755,462, filed Sep. 4, 1991; and U.S. Ser. No. 07/520,321, filed May 7, 1990, each incorporated herein by reference. The procedures below are directed to the use of E. coli recA, although as will be appreciated by those in the art, other recombinases may be used as well. Targeting polynucleotides can be coated using GTP_{Y}S, mixes of ATP_{Y}S with rATP, rGTP and/or dATP, or dATP or rATP alone in the presence of an rATP generating system (Boehringer Mannheim). Various mixtures of GTP_{Y}S, ATP_{Y}S, ATP, ADP, dATP and/or rATP or other nucleosides may be used, particularly preferred are mixes of ATP_{Y}S and ATP or ATP_{Y}S and ADP.

RecA protein coating of targeting polynucleotides is typically carried out as described in U.S. Ser. No. 07/910,791, filed July 9, 1992 and U.S. Ser. No. 07/755,462, filed Sep. 4, 1991, which are incorporated herein by reference. Briefly, the targeting polynucleotide, whether double-stranded or single-stranded, is denatured by heating in an aqueous solution at 95-100° C. for five minutes, then placed in an ice bath for 20 seconds to about one minute followed by centrifugation at 4° C. for approximately 20 sec, before use. When denatured targeting polynucleotides are not placed in a freezer at −20° C. they are usually immediately added to standard recA coating reaction buffer containing ATP_{Y}S, at room temperature, and to this is added the recA protein. Alternatively, recA protein may be included with the buffer components and ATP_{Y}S before the polynucleotides are added.

RecA coating of targeting polynucleotide(s) is initiated by incubating polynucleotide-recA mixtures at 37° C. for 10-15 min. RecA protein concentration tested during reaction with polynucleotide varies depending upon polynucleotide size and the amount of added polynucleotide, and the ratio of recA molecule:nucleotide preferably ranges between about 3:1 and 1:3. When single-stranded polynucleotides are recA coated independently of their homologous polynucleotide strands, the mM and pM concentrations of ATPyS and recA, respectively, can be reduced to one-half those used with double-stranded targeting polynucleotides (i.e., recA and ATP_{Y}S concentration ratios are usually kept constant at a specific concentration of individual polynucleotide strand, depending on whether a single- or double-stranded polynucleotide is used).

RecA protein coating of targeting polynucleotides is normally carried out in a standard 1X RecA coating reaction buffer. 10X RecA reaction buffer (i.e., 10x AC buffer) consists of: 100 mM Tris acetate (pH 7.5 at 37° C.), 20 mM magnesium acetate, 500 mM sodium acetate, 10 mM DTT, and 50% glycerol). All of the targeting polynucleotides, whether double-stranded or single-stranded, typically are denatured before use by heating to 95-100° C. for five minutes, placed on ice for one minute, and subjected to centrifugation (10,000 rpm) at 0° C. for approximately 20 seconds (e.g., in a Tomy centrifuge). Denatured targeting polynucleotides usually are added immediately to room temperature RecA coating reaction buffer mixed with ATP_{Y}S and diluted with buffer or double-distilled H₂O as necessary.

A reaction mixture typically contains the following components: (i) 0.2-4.8 mM ATP_{Y}S; and (ii) between 1-100 ng/μl of targeting polynucleotide. To this mixture is added about 1-20 μl of recA protein per 10-100 ml of reaction mixture, usually at about 2-10 mg/ml (purchased from Pharmacia or purified), and is rapidly added and mixed. The final reaction volume-for RecA coating of targeting polynucleotide is usually in the range of about 10-500 μl. RecA coating of targeting polynucleotide is usually initiated by incubating targeting polynucleotide-RecA mixtures at 37° C. for about 10-15 min.

RecA protein concentrations in coating reactions varies depending upon targeting polynucleotide size and the amount of added targeting polynucleotide: recA protein concentrations are typically in the range of 5 to 50 μM. When single-stranded targeting polynucleotides are coated with recA, independently of their complementary strands, the concentrations of ATP_{Y}S and recA protein may optionally be reduced to about one-half of the concentrations used with double-stranded targeting polynucleotides of the same length: that is, the recA protein and ATP_{Y}S concentration ratios are generally kept constant for a given concentration of individual polynucleotide strands.

The coating of targeting polynucleotides with recA protein can be evaluated in a number of ways. First, protein binding to DNA can be examined using band-shift gel assays (McEntee et al., (1981) J. Biol. Chem. 256: 8835). Labeled polynucleotides can be coated with recA protein in the presence of ATP_{Y}S and the products of the coating reactions may be separated by agarose gel electrophoresis.

Following incubation of recA protein with denatured duplex DNAs the recA protein effectively coats single-stranded targeting polynucleotides derived from denaturing a duplex DNA. As the ratio of recA protein monomers to nucleotides in the targeting polynucleotide increases from 0, 1:27, 1:2.7 to 3.7:1 for 121-mer and 0, 1:22, 1:2.2 to 4.5:1 for 159-mer, targeting polynucleotide's electrophoretic mobility decreases, i.e., is retarded, due to recA-binding to the targeting polynucleotide. Retardation of the coated polynucleotide's mobility reflects the saturation of targeting polynucleotide with recA protein. An excess of recA monomers to DNA nucleotides is required for efficient recA coating of short targeting polynucleotides (Leahy et al., (1986) J. Biol. Chem. 261: 954).

A second method for evaluating protein binding to DNA is in the use of nitrocellulose filter binding assays (Leahy et al., (1986) J. Biol. Chem. 261:6954; Woodbury, et al., (1983) Biochemistry 22(20):4730-4737. The nitrocellulose filter binding method is particularly useful in determining the dissociation-rates for protein:DNA complexes using labeled DNA. In the filter binding assay, DNA:protein complexes are retained on a filter while free DNA passes through the filter. This assay method is more quantitative for dissociation-rate determinations because the separation of DNA:protein complexes from free targeting polynucleotide is very rapid.

Alternatively, recombinase protein(s) (prokaryotic, eukaryotic or endogeneous to the target cell) may be exogenously induced or administered to a target cell simultaneously or contemporaneously (i.e., within about a few hours) with the targeting polynucleotide(s). Such administration is typically done by microinjection, although electroporation, lipofection, and other transfection methods known in the art may also be used. Alternatively, recombinase-proteins may be produced in vivo. For example, they may be produced from a homologous or heterologous expression cassette in a transfected cell or transgenic cell, such as a transgenic totipotent cell (e.g. a fertilized zygote) or an embryonal stem cell (e.g., a murine ES cell such as AB-1) used to generate a transgenic non-human animal line or a somatic cell or a pluripotent hematopoietic stem cell for reconstituting all or part of a particular stem cell population (e.g. hematopoietic) of an individual. Conveniently, a heterologous expression cassette includes a modulatable promoter, such as an ecdysone-inducible promoter-enhancer combination, an estrogen-induced promoter-enhancer combination, a CMV promoter-enhancer, an insulin gene promoter, or other cell-type specific, developmental stage-specific, hormone-inducible, or other modulatable promoter construct so that expression of at least one species of recombinase protein from the cassette can by modulated for transiently producing recombinase(s) in vivo simultaneous or contemporaneous with introduction of a targeting polynucleotide into the cell. When a hormone-inducible promoter-enhancer combination is used, the cell must have the required hormone receptor present, either naturally or as a consequence of expression a co-transfected expression vector encoding such receptor. Alternatively, the recombinase may be endogeneous and produced in high levels. In this embodiment, preferably in eukaryotic target cells such as tumor cells, the target cells produce an elevated level of recombinase. In other embodiments the level of recombinase may be induced by DNA damaging agents, such as mitomycin C, UV or gamma-irradiation. Alternatively, recombinase levels may also be elevated by transfection of a virus or plasmid encoding the recombinase gene into the cell.

A targeting polynucleotide of the invention may optionally be conjugated, typically by covalently or preferably noncovalent binding, to a cell-uptake component. As used herein, the term “cell-uptake component” refers to an agent which, when bound, either directly or indirectly, to a targeting polynucleotide, enhances the intracellular uptake of the targeting polynucleotide into at least one cell type (e.g., hepatocytes). A cell-uptake component may include, but is not limited to, the following: specific cell surface receptors such as a galactose-terminal glycoprotein (asialoorosomucoid (ASOR)) capable of being internalized into hepatocytes via a hepatocyte asialoglycoprotein receptor, a polycation (e.g., poly-L-lysine), and/or a protein-lipid complex formed with the targeting polynucleotide. Various combinations of the above ((ASOR)-poly-L-lysine), as well as alternative cell-uptake components will be apparent to those of skill in the art and are provided in the published literature (Wu GY and Wu CH (1987) J. Biol. Chem. 262:4429; Wu GY and Wu CH (1988) Biochemistry 27:887; Wu GY and Wu CH (1988) J. Biol. Chem. 263:14621; Wu GY and Wu CH (1992) J. Biol. Chem. 267: 12436; Wu et al. (1991) J. Biol. Chem. 266: 14338; and Wilson et al. (1992) J. Biol. Chem. 267: 963, WO92/06180; WO92/05250; and WO91/17761, which are incorporated herein by reference).

Alternatively, a cell-uptake component may be formed by incubating the targeting polynucleotide with at least one lipid species and at least one protein species to form protein-lipid-polynucleotide complexes consisting essentially of the targeting polynucleotide and the lipid-protein cell-uptake component. Lipid vesicles made according to Felgner (W091/17424, incorporated herein by reference) and/or cationic lipidization (WO91/16024, incorporated herein by reference) or other forms for polynucleotide administration (EP 465,529, incorporated herein by reference) may also be employed as cell-uptake components. Nucleases may also be used.

In addition to cell-uptake components, targeting components such as nuclear localization signals may be used, as is known in the art.

In addition to recombinase and cellular uptake components, the targeting polynucleotides may include chemical substituents. Exogenous targeting polynucleotides that have been modified with appended chemical substituents may be introduced along with recombinase (e.g., recA) into a target cell to homologously pair with a predetermined endogenous DNA target sequence in the cell. In a preferred embodiment, the exogenous targeting polynucleotides are derivatized, and additional chemical substituents are attached, either during or after polynucleotide synthesis, respectively, and are thus localized to a specific endogenous target sequence where they produce an alteration or chemical modification to a local DNA sequence. Preferred attached chemical substituents include, but are not limited to: cross-linking agents (see Podyminogin et al., Biochem. 34:13098 (1995) and 35:7267 (1996), both of which are hereby incorporated by reference), nucleic acid cleavage agents, metal chelates (e.g., iron/EDTA chelate for iron catalyzed cleavage), topoisomerases, endonucleases, exonucleases, ligases, phosphodiesterases, photodynamic porphyrins, chemotherapeutic drugs (e.g., adriamycin, doxirubicin), intercalating agents, labels, base-modification agents, agents which normally bind to nucleic acids such as labels, etc. (see for example Afonina et al., PNAS USA 93:3199 (1996), incorporated herein by reference) immunoglobulin chains, and oligonucleotides. Iron/EDTA chelates are particularly preferred chemical substituents where local cleavage of a DNA sequence is desired (Hertzberg et al. (1982) J. Am. Chem. Soc. 104: 313; Hertzberg and Dervan (1984) Biochemistry 23:

3934; Taylor et al. (1984) Tetrahedron 40: 457; Dervan, P B ( 1986) Science 232: 464, which are incorporated herein by reference). Further preferred are groups that prevent hybridization of the complementary single stranded nucleic acids to each other but not to unmodified nucleic acids; see for example Kutryavin et al., Biochem. 35:11170 (1996) and Woo et al., Nucleic Acid. Res. 24(13):2470 (1996), both of which are incorporated by reference. 2′-O methyl groups are also preferred; see Cole-Strauss et al., Science 273:1386 (1996); Yoon et al., PNAS 93:2071 (1996)). Additional preferred chemical substitutents include labeling moieties, including fluorescent labels. Preferred attachment chemistries include: direct linkage, e.g., via an appended reactive amino group (Corey and Schultz (1988) Science 238:1401, which is incorporated herein by reference) and other direct linkage chemistries, although streptavidin/biotin and digoxigenin/antidigoxigenin antibody linkage methods may also be used. Methods for linking chemical substituents are provided in U.S. Pat. Nos. 5,135,720, 5,093,245, and 5,055,556, which are incorporated herein by reference. Other linkage chemistries may be used at the discretion of the practitioner.

Typically, a targeting polynucleotide of the invention is coated with at least one recombinase and is conjugated to a cell-uptake component, and the resulting cell targeting complex is contacted with a target cell under uptake conditions (e.g., physiological conditions) so that the targeting polynucleotide and the recombinase(s) are internalized in the target cell. A targeting polynucleotide may be contacted simultaneously or sequentially with a cell-uptake component and also with a recombinase; preferably the targeting polynucleotide is contacted first with a recombinase, or with a mixture comprising both a cell-uptake component and a recombinase under conditions whereby, on average, at least about one molecule of recombinase is noncovalently attached per targeting polynucleotide molecule and at least about one cell-uptake component also is noncovalently attached. Most preferably, coating of both recombinase and cell-uptake component saturates essentially all of the available binding sites on the targeting polynucleotide. A targeting polynucleotide may be preferentially coated with a cell-uptake component so that the resultant targeting complex comprises, on a molar basis, more cell-uptake component than recombinase(s). Alternatively, a targeting polynucleotide may be preferentially coated with recombinase(s) so that the resultant targeting complex comprises, on a molar basis, more recombinase(s) than cell-uptake component.

Cell-uptake components are included with recombinase-coated targeting polynucleotides of the invention to enhance the uptake of the recombinase-coated targeting polynucleotide(s) into cells for gene targeting applications, such as the production of transgenic organisms as described herein. Alternatively, a targeting polynucleotide may be coated with the cell-uptake component and targeted to cells with a contemporaneous or simultaneous administration of a recombinase (e.g., liposomes or immunoliposomes containing a recombinase, a viral-based vector encoding and expressing a recombinase).

Once the recombinase-targeting polynucleotide compositions are formulated, they are introduced or administered into target cells. In a preferred embodiment, the targeting polynucleotides are used to alter a chromosomal sequence of a donor nucleus of a donor (target) cell. By “donor nucleus” herein is meant a nucleus of a donor or target cell. The administration is typically done as is known for the administration of nucleic acids into cells, and, as those skilled in the art will appreciate, the methods may depend on the choice of the target cell. Suitable methods include, but are not limited to, microinjection, piezo-driven micropipette injection, electroporation, lipofection, biolistics, chemical treatment of cells etc.

By “target cell” or “donor cell” and grammatical equivalents herein is meant a cell, preferably eukaryotic, that comprises a predetermined target sequence. Suitable eukaryotic cells include, but are not limited to, plant cells including those of corn, sorghum, tobacco, canola, soybean, cotton, tomato, potato, alfalfa, sunflower, etc.; and animal cells, including fish, birds and mammals. Suitable fish cells include, but are not limited to, those from species of salmon, trout, tulapia, tuna, carp, flounder, halibut, swordfish, cod and zebrafish. Suitable bird cells include, but are not limited to, those of chickens, ducks, quail, pheasants and turkeys, and other jungle fowl or game birds. Suitable mammalian cells include, but are not limited to, cells from horses, cattle, buffalo, ungulates, deer, sheep, rabbits, rodents such as mice, rats, hamsters, gerbils, and guinea pigs, minks, goats, pigs, primates, marsupials, marine mammals including dolphins and whales, as well as cell lines, such as human cell lines, of any tissue or stem cell type, and stem cells, including pluripotent and non-pluripotent, and non-human zygotes, although making trangenic humans is not preferred. The cells can be haploid, diploid, an embryonal cell (i.e., embryonal germ cell, embryonal stem cell, an endodermal cell, a mesodermal cell, an ectodermal cell, a neural crest cell, a neural crest stem cell), a fetal cell (i.e., an umbilical cord cell, an umbilical cord blood cell), a somatic cell (i.e. a mammary derived cell, an adult tail-tip cell, a cumulus cell, an epithelial cell, a dermal cell, a keratinocyte, a melanocyte, a mesenchymal cell, a stem cell, a blood cell, a fibroblast) or non-somatic, i.e., germinal cells (germ cell, a germ cell precursor, a germ stem cell) or gametocytes.

In one embodiment the donor cell is a somatic cell of a eukaryotic organism. By “somatic cell” herein is meant any cell of an organism, fetus, or an embryo that is not a “germ cell”. In a preferred embodiment for making transgenic nonhuman animals, the donor cell is preferably a eukaryotic somatic cell. In this embodiment, a pre-selected target DNA sequence is chosen for alteration. Preferably, the pre-selected target DNA sequence is a chromosomal sequence. By “chromosomal sequence” herein is meant a sequence that is contained within the chromosome or genomic sequences. Preferred chromosomal sequences include sequences encoding open reading frames or HMTs (homology motif tags), exons, introns, transcriptional regulatory regions, highly repetative sequences, a provirus, transpositional element, sequences of unknown function etc. As described herein, a recombinase and at least two single stranded targeting polynucleotides which are substantially complementary to each other, each of which contain a homology clamp to the target sequence contained on the chromosomal sequence, are added to the target cell, preferably in vitro. The two single stranded targeting polynucleotides are preferably coated with a recombinase, and at least one of the targeting polynucleotides contain at least one nucleotide substitution, insertion or deletion or any combination thereof. The targeting polynucleotides then bind to the target sequence in the chromosomal sequence to effect homologous recombination and form an altered chromosomal sequence which contains the substitution, insertion and/or deletion. In this embodiment, it may be desirable to bind (generally non-covalently) a nuclear localization signal to the targeting polynucleotides to facilitate localization of the complexes in the nucleus. See for example Kido et al., Exper. Cell Res. 198:107-114 (1992), hereby expressly incorporated by reference. The targeting polynucleotides and the recombinase function to effect homologous recombination, resulting in altered chromosomal or genomic sequences.

In other embodiments, somatic cells are used, such as fetal fibroblasts (Cibelli et al. Science. 280:1256-1257(1998); Schnieke et al. Science. 278:2130-2133 (1997); Baguisi et al. Nature Biotechnology. 17:456-461 (1999)); oviductal epithelial cells (Kato et al. Science. 282:2095-2098 (1998)), cumulus cells from ovarian oocytes (Wakayama et al. Nature 394:369 (1998)); a mammary-derived cell (Wilmut et al. Nature. 385:810-813 (1997)); murine adult tail-tip cells (Wakayama et al. Nature Genetics. 22:127-128 (1999)). Suitable somatic cells are found in a number of animals, including fish, birds, and mammals. Somatic cells from suitable fish include, but are not limited to, those from species of salmon, trout, tuna, carp, flounder, halibut, swordfish, cod, medaka, tulapia and zebrafish. Suitable bird somatic cells include, but are not limited to, those of chickens, ducks, quail, pheasant, turkeys, and other jungle fowl and game birds. Suitable mammalian somatic cells include, but are not limited to, cells from horses, cattle, buffalo, deer, sheep, rabbits, rodents such as mice, rats, hamsters and guinea pigs, goats, pigs, primates, and marine mammals including dolphins and whales.

In a preferred embodiment, a somatic cell is diploid, that is having two of each chromosome characteristic of a given organism, the total number being twice that of a gamete. In alternative embodiments, the somatic cells are haploid, hypoploid or hyperploid relative to the number of chromosomes characteristic of the organism from which they originate.

In a preferred embodiment, the donor cell is a germ cell. By “germ cell” herein is meant a cell such as a gametocyte or a reproductive cell or a progenitor of a reproductive cell, for example, a germ cell stem cell. For example, a germ cell includes an oocyte or a spermatozoa that unite to form a cell that develops into a new individiual. By “oocyte” and “ovum” and grammatical equivalents herein are meant a female gamete. By “spermatzoa” and “spermatocyte” and grammatical equivalents herein are meant a male germ cell or gamete and fragments thereof, with the head of the spermatozoa being preferred.

In a preferred embodiment a germ cell is haploid, that is having one of each chromosome characteristic of a given organism, the total number being half that of a somatic cell. In alternative embodiments, the germ cells are aneuploid, hypoploid or hyperploid relative to the number chromosomes characteristic of the organism from which they originate.

In a preferred embodiment, the nucleus of an altered donor cell is removed and transplanted into a recipient cell, and used in the production of a recombinant organism using techniques well known in the art (Wilmut et al. 1997. Nature 385:810; WO99/35906; WO/9829532; WO99/01164; WO97/07669; WO97/07668; WO98/07841; WO98/30683; WO98/37183; WO98/39416; WO99/01163; WO99/47642; WO99/37143; WO99/36510; WO99/46982; WO99/05266; WO99/21415; U.S. Pat. No. 5,945,577; U.S. Pat. No. 5,907,080;, Baguisi et al. 1999. Nature Biotechnology. 17:456-461; Wakayama et al. 1999. Nature Genetics 22:127-128; Cibelli et al. 1998. Science 280:1256-1258; Kato et al. 1998. Science 282:2095-2099; Wakayama et al. 1998. Nature 394:369-374; Schnieke et al. 1997. Science. 278:2130-2133; Kono et al. 1991. J. Reprod. Fertil. 93(1):165-72; Le Bourhis et al. 1998. J. Reprod. Fertil. 113(2):343-8; McGrath et al. 1983. J. Exp. Zool. 228(2):355-62; McGrath et al. 1983. Science 220:1300-2; McLaughlin et al. 1990. Reprod. Fertil. Dev. 2(6):619-22; Meng et al. 1997. Biol. Reprod. 57(2):454-9; Prather et al. 1989. Biol. Reprod. 37(4):859-66; Prather et al. 1989. Biol. Reprod. 41(3):414-8; Robl et al. 1987. J. Anim. Sci. 64(2):642-7; Sims et al. 1994. PNAS USA 91(13):6143-7; Smith et al. 1989. Biol. Reprod. 40(5):1027-1035; Stice et al. 1988. Biol. Reprod. 39(3):657-64; Vignon et al. 1998. CR Acad Sci III. 321(9):735-45; Wells et al. 1997. Biol. Reprod. 57(2):385-93; Wells et al. 1999. Biol. Reprod. 60(4):996-1005; Wilmut et al. Nature 1997 Mar 13; 386(6621):200 and Nature 385(6619):810-3; Yang et al. 1992. Biol. Reprod. 47(4):636-43; campbell et al. Nature 38-(6569):64-6; Cheong et al. 1992. Jpn J. Vet. Res. 40(4):149-159; Cheong et al. 1993. Biol. Reprod. 48(5):958-63; Cibelli et al. 1998. Nature Biotechnology 16(7):642-6; First et al. 1992. J. Reprod. Fertil. Suppl. 43:245-54; Yong et al. 1998. Biol. Reprod. 58(1):266-9; Zakhartchenko et al. 1999. Mol. Reprod. Dev. 52(4):421-6, all of which are hereby incorporated by reference in their entirety)

In a preferred embodiment, suitable recipient cells include animal cells, including fish, birds and mammals. Suitable fish cells include, but are not limited to, those from species of salmon, trout, tulapia, tuna, carp, flounder, halibut, swordfish, cod and zebrafish. Suitable bird cells include, but are not limited to, those of chickens, ducks, quail, pheasants and turkeys, and other jungle fowl or game birds. Suitable mammalian cells include, but are not limited to, cells from horses, cattle, buffalo, deer, sheep, rabbits, rodents such as mice, rats, hamsters, gerbils, and guinea pigs, minks, goats, pigs, primates, marsupials, marine mammals including dolphins and whales, as well as cell lines, such as human cell lines, of any tissue or stem cell type, and stem cells.

In a preferred embodiment, the recipient cell is an oocyte, preferably an enucleated oocyte. By “enucleated oocyte” is meant an oocyte with the nucleus removed or destroyed. Preferred oocytes are those from a wide variety of organisms, with mammalian oocytes being preferred. Preferred oocytes are those from goats, cattle, minks, pigs, rodents (mice, rats, hamsters, guinea pigs, etc.), primates, plants, insects, reptiles, birds, fish, amphibians, crustaceans, molluscs etc. In general, human oocytes may not be preferred.

In a preferred embodiment, the recipient cell is an enucleated embryonic stem (ES) cell or an embryonic germ (EG) cell. Thus, in a preferred embodiment for making transgenic non-human animals (which include homologously targeted non-human animals) embryonal stem cells (ES cells) are preferred. Murine ES cells, such as AB-1 line grown on mitotically inactive SNL76/7 cell feeder layers (McMahon and Bradley, Cell 62: 1073-1085 (1990)) essentially as described (Robertson, E. J. (1987) in Teratocarcinomas and Embryonic Stem Cells: A Practical Approach. E. J. Robertson, ed. (oxford: IRL Press), p. 71-112; Zjilstra et al., Nature 342:435-438 (1989); and Schwartzberg et al., Science 246:799-803 (1989), each of which is incorporated herein by reference) may be used for homologous gene targeting. Other suitable ES lines include, but are not limited to, the E14 line (Hooper et al. (1987) Nature 326: 292-295), the D3 line (Doetschman et al. (1985) J. Embryol. Exp. Morph. 87: 21-45), and the CCE line (Robertson et al. (1 986) Nature 323: 445-448). The success of generating a mouse line from ES cells bearing a specific targeted mutation depends on the pluripotence of the ES cells (i.e., their ability, once injected into a host blastocyst or enucleated oocyte, to participate in embryogenesis and contribute to the germ cells of the resulting animal).

The pluripotence of any given ES or EG cell line can vary with time in culture and the care with which it has been handled. The only definitive assay for pluripotence is to determine whether the specific population of ES cells to be used can give rise to chimeras capable of germline transmission of the ES genome. For this reason, prior to gene targeting, a portion of the parental population of AB-1 cells is injected into C57B1/6J blastocysts to ascertain whether the cells are capable of generating chimeric mice with extensive ES cell contribution and whether the majority of these chimeras can transmit the ES genome to progeny.

The methods of the present invention are used to make recombinant zygotes. By “recombinant zygote” herein is meant a zygote produced according to the methods of the present invention. Accordingly, in one embodiment a “recombinant zygote” is formed by the introduction of a nucleus of a somatic cell into an enucleated oocyte. In another embodiment a “recombinant zygote” is formed by the injection of a spermatocyte into an oocyte. In another embodiment, a “recombinant zygote” is formed by introduction of haploid nucleus into an oocyte. In another embodiment a “recombinant zygote” is a zygote that has undergone enhanced homologous recombination according to the methods described herein. Accordingly, in one embodiment, a recombinant zygote, comprises a recombinant nucleic acid.

By “recombinant nucleic acid” herein is meant nucleic acid, originally formed in vitro or in a cell, in general, by the manipulation of nucleic acid by endonucleases and/or polymerase and/or recombinases and/or ligases to be in a form not normally found in nature. It is understood that once a recombinant nucleic acid is made and introduced into a host cell or organism, it will replicate non-recombinantly, i.e. using the in vivo cellular machinery of the host cell rather than in vitro manipulations; however, such nucleic acids, once produced recombinantly, although subsequently replicated non-recombinantly, are still considered recombinant for the purposes of the invention. In accordance with this definition, a cell, a cell organelle, a tissue, or organism or progeny thereof that comprises the recombinant nucleic acid also is considered to be a recombinant cell, organelle etc. Accordingly, in a preferred embodiment a recombinant nucleic acid comprises a transgene.

By “activated zygote” herein is meant a recombinant zyote, which has been stimulated in vitro to divide to form an embryo, morula, and/or blastocyst as is known in the art (Wilmut et al. 1997. Nature 385:810; WO99/35906; WO/9829532; WO99/01164; WO97/07669; WO97/07668; WO98/07841; WO98/30683; WO98/37183; WO98/39416; WO99/01163; WO99/47642; WO99/37143; WO99/36510; WO99/46982; WO99/05266; WO99/21415, U.S. Pat. No. 5,907,080, U.S. Pat. No. 5,945,577, Baguisi et al. 1999. Nature Biotechnology. 17:456-461; Wakayama et al. 1999. Nature Genetics 22:127-128; Cibelli et al. 1998. Science 280:1256-1258; Kato et al. 1998. Science 282:2095-2099; Wakayama et al. 1998.

Nature 394:369-374; Schnieke et al. 1997. Science. 278:2130-2133 all of which are hereby incorporated by reference in their entirety).

In a preferred embodiment, a zygote is activated for example by electroactivation or by contact with a chemical activator. Preferred chemical activators include, Ca²⁺ release stimulators, Ca²⁺ ionophores, strontium ions, sperm cytoplasmic factors, inhibitors of protein synthesis, oocyte receptor ligand mimetics, regulators of phosphoprotein signaling, and ethyl alcohol.

The methods herein are used to make transgenic organisms. By the term, “transgenic organism” or “recombinant organism” and grammatical equivalents herein is meant a plant or animal having at least one cell that contains a transgene, which transgene in a preferred embodiment was introduced into the organism or an ancestor of the organism at a prenatal stage, for example, at the embryonic or zygote stage or introduced into a gametocyte. In one embodiment, the transgene is foreign to the organism. In another embodiment, the transgene is native to the organism, such as a transgene the corrects a disease allele. In yet another embodiment, the transgene is a non-naturally occuring form, such as, a disease allele, or is a naturally or non-naturally occurring form that is in a non-natural position in the genome of the transgenic organism. Accordingly, for purposes of the invention, a transgene modifies at least one nucleotide of its host organism. In a preferred embodiment, the transgene is passed onto the progeny of the transgenic organism. Preferably, the transgene modifies the phenotype of a transgenic organism or is expressed in at least one cell of an transgenic organism. Accordingly, a transgene is optionally expressed prenataly and/or after the birth and/or throughout the life of a transgenic organism. The transgene is optionally expressed in all cells or a subset of cells and is expressed either constitutively or in response to specific stimuli.

The term “naturally-occurring” as used herein as applied to an object refers to the fact that an object can be found in nature. For example, a polynucleotide sequence that is present in an organism (including viruses) that can be isolated from a source in nature and which has not been intentionally modified by man in the laboratory is naturally-occurring.

In a preferred embodiment, the donor or recipient cell are metabolically active. A “metabolically-active cell” is a cell, comprising an intact nucleoid or nucleus, which, when provided nutrients and incubated in an appropriate medium carries out DNA and/or RNA synthesis for extended periods (e.g., at least 12-24 hours). Such metabolically-active cells are typically undifferentiated or differentiated cells capable or incapable of further cell division (although non-dividing cells many undergo nuclear division and chromosomal replication), although stem cells and progenitor cells are also metabolically-active cells. Alternatively, donor or recipient cell are not metabolically active.

In an alternative embodiment, the donor nucleus or cell is metabolically inactive, for example, if it is to be fused with a metabolically active recipient cell or nucleus. In an alternative embodiment, neither donor or recipient are metabolically active, but are induced to be metabolically active by physical, chemical, biological or others means as known in the art.

Once targeting polynucleotides and recombinase has been introduced into the nucleus of a target cell, the nucleus is isolated and inserted into an enucleated oocyte to form a recombinant zygote, which is activated and transferred to surrogate mothers. In an alternative embodiment, the nucleus is first isolated from the target cell and the targeting polynucleotides and recombinase are introduced. In yet another embodiment, the nucleus is removed from the target cell and inserted into an enucleated oocyte followed by the introduction of targeting polynucleotides and recombinase. (see Kimura et al. Development. 121:2397-2405 (1995); Cibelli et al. Science 280:1256-1258 (1998); Campbell et al. Nature. 380:64 (1996); Wilmut et al. Nature. 385:810 (1997); Baguisi et al. Nature Biotechnology 17:456-461 (1999); Wakayama et al. Nature 394:369-374, and Kato et al. Science, and references cited above, all expressly incorporated by references. Optionally, the nuclei may be cryopreserved prior to transplantation as known in the art at the convenience of the practitioner.

In another preferred embodiment, transgenic organisms are produced by co-injection of oocytes with spermatozoa (Kimura et al. Biology of Reproduction 52:709-720 (1995); Perry et al. Science 284:1180 (1999), targeting polynucleotides and a recombinase to produce a recombinant zygote. The recombinant zygote is activated and transplanted into surrogate mothers. In a preferred embodiment, spermatocytes are membrane disrupted by freeze-thaw (Wakayama et al. J. Fertil. Reprod. 112:11 (1998)), lyophilization (Wakayama et al. Nature Biotechnol. 16:639: (1998)) and re-hydrated, or detergent treatment (Perry et al. Science 284:1180 (1999)). Without being bound by theory, membrane disruption exposes basic proteins in the perinuclear matrix that reversibly bind to the negatively charged targeting polynucleotides or nucleoprotein filaments. Accordingly, the targeting polynucleotides and recombinase are preferably associated prior to intracytoplasmic injection. The membrane-disrupted spermatocytes act as a vehicle for the introduction of targeting polynucleotides, recombinase and nuceloprotein filaments into oocytes. Intracytoplasmic injection is preferably by a piezo-driven micropipette.

In another embodiment, transgenic animals are produced by targeting and altering a preselected target sequence in a non-human, recombinant or non-recombinant zygote, for example, using techniques known in the art (see U.S. Pat. No. 4,873,191; Brinster et al., PNAS 86:7007 (1989); Susulic et al., J. Biol. Chem. 49:29483 (1995), and Cavard et al., Nucleic Acids Res. 16:2099 (1988), hereby incorporated by reference.). Preferred zygotes include, but are not limited to, animal zygotes, including fish, avian and mammalian zygotes. Suitable fish zygotes include, but are not limited to, those from species of salmon, trout, tuna, carp, flounder, halibut, swordfish, cod, tulapia and zebrafish. Suitable bird zygotes include, but are not limited to, those of chickens, ducks, quail, pheasant, turkeys, and other jungle fowl and game birds. Suitable mammalian zygotes include, but are not limited to, cells from horses, cattle, buffalo, deer, sheep, rabbits, rodents such as mice, rats, hamsters and guinea pigs, goats, pigs, primates, and marine mammals including dolphins and whales. See Hogan et al., Manipulating the Mouse Embryo (A Laboratory Manual), 2nd Ed. Cold Spring Harbor Press, 1994, incorporated by reference. Following introduction of targeting polynucleotides and recombinase, the zygote is activated and introduced into a surrogate mother.

In general, transgenic animals are made with any number of changes. Exogeneous sequences, or extra copies of endogeneous sequences, including structural genes and regulatory sequences, may be added to the animal, as outlined below. Endogeneous sequences (again, either genes or regulatory sequences) may be disrupted, i.e. via insertion, deletion or substitution, to prevent expression of endogeneous proteins. Alternatively, endogeneous sequences may be modified to alter their biological function, for example via mutation of the endogeneous sequence by insertion, deletion or substitution.

Accordingly, the methods of the present invention are useful to add exogenous DNA sequences, such as exogenous genes or regulatory sequences, extra copies of endogenous genes or regulatory sequences, or exogeneous genes or regulatory sequences, to a transgenic plant or animal. This may be done for a number of reasons: for example, adding one or more copies of a wild-type gene can increase the production of a desirable gene product; adding or deleting one or more copies of a therapeutic gene can alleviate a disease state, or to create an animal model of disease. Adding one or more copies of a modified wild type gene may be done for the same reasons. Adding therapeutic genes or proteins may yield superior transgenic animals, for example for the production of therapeutic or nutriceutical proteins. Adding human genes to non-human mammals may facilitate production of human proteins and adding regulatory sequences derived from human or non-human mammals may be useful to increase or decrease the expression of endogenous or exogenous genes. Such inserted genes may be under the control of endogenous or exogenous regulatory sequences, as described herein.

The methods of the invention are also useful to modify endogeneous gene sequences, as outlined below. Suitable endogenous gene targets include, but are not limited to, genes which encode peptides or proteins including enzymes, structural or soluble proteins, as well as endogeneous regulatory sequences including, but not limited to, promoters, transcriptional or translational sequences, repetitive sequencs including oligo[d(A-C)ₙ •d(G-T)ₙ], oligo[d(A-T)]ₙ, oligo[d(C-T)]ₙ, etc. Examples of such endogenous gene targets include, but are not limited to, pigment genes, DNA repair genes, DNA replication genes, cell cycle control genes, mitochondrial genes, chloroplast genes, growth genes, hormone genes, apoptosis genes, senescence genes, neurotrophic factor genes, genes which encode lactoglobulins including both α-lactoglobulin and β-lactoglobulin; casein, including both α-casein, β-casein and κ-casein; albumins, including serum albumin, particularly human and bovine; immunoglobulins, including IgE, IgM, IgG and IgD and monoclonal antibodies; globin; integrin; hormones; growth factors, particularly bovine and human growth factors, including transforming growth factor, epidermal growth factor, nerve growth factors, etc.; collagen; interleukins, including IL-1 to IL-17; a major histocompatibility antigen (MHC); G-protein coupled receptors (GPCR); nuclear receptors; ion channels; multidrug resistance genes; amyloid proteins; enzymes, including esterases, proteases (including tissue plasminogen activator (tPA)), lipases, carbohydrases, etc.; APRT, HPRT; leptin; tumor suppressor genes; provirus; prions; OTC; CFTR; sugar transferases such as alpha-galactosyl transferase (gaIT) or fucosyl transferase; a milk or urine protein gene including the caseins, lactoferrin and whey proteins; oncogenes; cytokines, particularly human; transcription factors; and other pharmaceuticals. Any or all of these may also be suitable exogeneous genes to add to a genome using the methods outlined herein.

Endogeneous genes (or regulatory sequences, as outlined herein) may be modified in several ways, including disruptions and alterations.

The endogenous target gene may be disrupted in a variety of ways. The term “disrupt” as used herein comprises a change in the coding or non-coding sequence of an endogenous nucleic acid that alters the transcription or translation of an endogenous gene. In a preferred embodiment, a disrupted gene will no longer produce a functional gene product. Generally, disruption may occur by either the insertion, deletion or frame shifting of nucleotides.

The term “insertion sequence” as used herein means one or more nucleotides which are inserted into an endogenous gene to disrupt it. In general, insertion sequences can be as short as 1 nucleotide or as long as a gene, as outlined below. For non-gene insertion sequences, the sequences are at least 1 nucleotide, with from about 1 to about 50 nucleotides being preferred, and from about 10 to 25 nucleotides being particularly preferred. An insertion sequence may comprise a polylinker sequence, with from about 1 to about 50 nucleotides being preferred, and from about 10 to 25 nucleotides being particularly preferred.

In a preferred embodiment, an insertion sequence comprises a gene which not only disrupts the endogenous gene, thus preventing its expression, but also can result in the expression of a new gene product. Thus, in a preferred embodiment, the disruption of an endogenous gene by an insertion sequence gene is done in such a manner to allow the transcription and translation of the insertion gene. An insertion sequence that encodes a gene may range from about 50 bp to 5000 bp of cDNA or about 5000 bp to 50000 bp of genomic DNA. As will be appreciated by those in the art, this can be done in a variety of ways. In a preferred embodiment, the insertion gene is targeted to the endogenous gene in such a manner as to utilize endogenous regulatory sequences, including promoters, enhancers or a regulatory sequence. In an alternate embodiment, the insertion sequence gene includes its own regulatory sequences, such as a promoter, enhancer or other regulatory sequence etc.

Particularly preferred insertion sequence genes include, but are not limited to, genes which encode therapeutic and nutriceutical proteins, and reporter genes. Suitable insertion sequence genes which may be inserted into endogenous genes include, but are not limited to, nucleic acids which encode those genes listed as suitable endogeneous genes for alterations, above, particularly mammalian enzymes, mammalian antibodies, mammalian proteins including serum albumin as well as mammalian therapeutic genes. In a preferred embodiment, the inserted mammalian gene is a human gene. Suitable reporter genes are those genes which encode detectable proteins, such as the genes encoding luciferase, β-galactosidase (both of which require the addition of reporter substrates), and the fluorescent proteins, including green fluorescent protein (GFP), blue fluorescent protein (BFP), yellow fluorescent protein (YFP), and red fluorescent protein (RFP).

Thus, in a preferred embodiment, the targeted sequence modification creates a sequence that has a biological activity or encodes a polypeptide having a biological activity. In a preferred embodiment, the polypeptide is an enzyme with enzymatic activity. In another preferred embodiment, the polypeptide is an antibody. In a third preferred embodiment, the polypeptide is a structural protein.

In addition, the insertion sequence genes may be modified or variant genes, i.e. they contain a mutation from the wild-type sequence. Thus, for example, modified genes including, but not limited to, improved therapeutic genes, modified a-lactalbumin genes that do not encode any phenylalanine residues, or human enzyme or human antibody genes that do not encode any phenylalanine residues.

The term “deletion” as used herein comprises removal of a portion of the nucleic acid sequence of an endogenous gene. Deletions range from about 1 to about 100 nucleotides, with from about 1 to 50 nucleotides being preferred and from about 1 to about 25 nucleotides being particularly preferred, although in some cases deletions may be much larger, and may effectively comprise the removal of the entire endogenous gene and/or its regulatory sequences. Deletions may occur in combination with substitutions or modifications to arrive at a final modified endogenous gene.

In a preferred embodiment, endogenous genes may be disrupted simultaneously by an insertion and a deletion. For example, some or all of an endogenous gene, with or without its regulatory sequences, may be removed and replaced with an insertion sequence gene. Thus, for example, all but the regulatory sequences of an endogenous gene may be removed, and replaced with an insertion sequence gene, which is now under the control of the endogenous gene's regulatory elements.

The term “regulatory element” is used herein to describe a non-coding sequence which affects the transcription or translation of a gene including, but are not limited to, promoter sequences, ribosomal binding sites, transcriptional start and stop sequences, translational start and stop sequences, enhancer or activator sequences, or dimerizing sequences. In a preferred embodiment, the regulatory sequences include a promoter and transcriptional start and stop sequence.

Promoter sequences encode either constitutive or inducible promoters. The promoters may be either naturally occurring promoters or hybrid promoters. Hybrid promoters, which combine elements of more than one promoter, are also known in the art, and are useful in the present invention.

In addition to disrupting endogeneous genes, the endogeneous genes may be altered by substitutions, insertions or deletions of nucleotides that do not completely eliminate the biological function of the sequence, but rather alter it. That is, targeted gene modifications may be made to alter gene function. For example, defective genes may be fixed, or the activity of a gene may be modulated, either increasing or decreasing the activity of the sequence (either the nucleic acid sequence, for example in the case of regulatory nucleic acid, or of the gene product, i.e. the amino acid sequence of the protein may be altered).

The methods of the present invention are useful to provide methods for fully or partially modifying endogenous regulatory sequences. Suitable targets for such fully or partially modified regulatory sequences include, but are not limited to, regulatory sequences that regulate any of the suitable endogeneous genes listed above, with preferred embodiments altering the endogeneous regulatory sequences that control the genes which encode α-lactoglobulin, β-lactoglobulin, casein, α-casein, β-casein, κ-casein, serum albumin, globin, IgG, integrin, lactoferrin, a retroviral provirus, a prion, a leptin, a hormone, a neurotrophin, alpha-galactosyl transferase (gaIT), a sugar transferase or a milk or urine production gene. Examples of such fully or partially modified endogenous regulatory sequences include, but are not limited to, a modified regulatory element for an endogenous gene, a modified transcriptional regulation cassette or start site for an endogenous gene, a modified promoter, transcription initiation site, or enhancer sequences.

When the modification of the endogeneous gene is to alter a structural gene, generally amino acid changes will be made as is known in the art. Substitutions, deletions, insertions or any combination thereof may be used to arrive at a final derivative. Generally these changes are done on a few amino acids to minimize the alteration of the molecule. However, larger changes may be tolerated in certain circumstances or for certain purposes. When small alterations in the characteristics of the endogeneous protein are desired, substitutions are generally made in accordance with the following chart:

Chart I

| Original Residue | Exemplary Substitutions |
|---|---|
| Ala | Ser |
| Arg | Lys |
| Asn | Gln, His |
| Asp | Glu |
| Cys | Ser |
| Gln | Asn |
| Glu | Asp |
| Gly | Pro |
| His | Asn, Gln |
| Ile | Leu, Val |
| Leu | Ile, Val |
| Lys | Arg, Gln, Glu |
| Met | Leu, Ile |
| Phe | Met, Leu, Tyr |
| Ser | Thr |
| Thr | Ser |
| Trp | Tyr |
| Tyr | Trp, Phe |
| Val | Ile, Leu |

Substantial changes in function or immunological identity are made by selecting substitutions that are less conservative than those shown in Chart I. For example, substitutions may be made which more significantly affect: the structure of the polypeptide backbone in the area of the alteration, for example the a-helical or b-sheet structure; the charge or hydrophobicity of the molecule at the target site; or the bulk of the side chain. The substitutions which in general are expected to produce the greatest changes in the polypeptide's properties are those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g. lysyl, arginyl, or histidyl, is substituted for (or by) an electronegative residue, e.g. glutamyl or aspartyl; or (d) a residue having a bulky side chain, e.g. phenylalanine, is substituted for (or by) one not having a side chain, e.g. glycine.

Preferred embodiments of the present invention include, but are not limited to: (1) a farm animal including cattle, sheep, pigs, horses and goats with a 1-25 base pair deletion, or a 10-25 base pair insertion of a polylinker sequence, or insertion of a reporter gene such as a luciferase gene, a β-galactosidase gene or a green fluorescent (GFP) protein gene in an endogenous gene or sequence encoding ornithine transcarbamylase (OTC), lactoglobulin, casein, β-casein, α-casein, κ-casein, albumin, globin, immunoglobulin, IgG, interleukin, a sugar transferase, integrin, a milk protein, a urine protein, a retroviral provirus, an endogenous virus, a prion, a leptin, or cystic fibrosis transmembrane regulator (CFTR); (2) a farm animal including cattle, sheep, pigs, horses and goats with an exogenous gene such as a gene encoding human lysozyme, human growth hormone, human serum albumin, human globin, a human antibody (human IgG), a tissue plasminogen activator, a human therapeutic protein, human lactase, a human lipase, a hormone receptor gene, a viral receptor gene, a G-protein coupled receptor gene, a drug or a human enzyme gene, including for example the human lysozyme gene, the human α-1 anti-trypsin gene, the human anti-thrombin IlI gene; (4) a farm animal including cattle, sheep, pigs, horses and goats with a modified endogenous repeated (A-C)ₙ sequence, a modified repeated (A-G)ₙ sequence, a modified repeated (A-T)ₙ sequence, a modified endogenous CFTR gene or a modified endogenous OTC gene; (5) a farm animal including cattle, sheep, pigs, horses and goats with a modified α-lactoglobulin gene or β-lactoglobulin gene does not encode any phenylalanine residues; (6) a farm animal including cattle, sheep, pigs, horses and goats with a human monoclonal antibody gene, or a gene for a human antibody that does not encode any phenylalanine residues, for example inserted (or replacing) in the endogenous gene or sequence encoding an immunoglobulin, or IgG; and (7) a farm animal including cattle, sheep, pigs, horses and goats with a human gene under control of its endogenous promoter, a modified endogenous regulatory element for an endogenous gene which may or may not be disrupted by an insertion sequence, a transcriptional regulation cassette ord a dimerizing sequence. Specific preferred embodiments also include, a farm animal including cattle, sheep, pigs, horses and goats with an endogenous regulatory element which is disrupted by, deletion of at least one nucleotide.

Additional preferred embodiments comprise a pig, monkey or cow with a 1-25 to 1-50 base pair insertion, examples of which include a hormone receptor gene, a viral receptor gene or a G-protein coupled receptor gene, or a 1-25 to 1-50 bp deletion in a sugar transferase gene including the α-galactosyl transferase gene (galT) or the fucosyl transferase gene, a BELE® goat with a human gene, and a pig, goat, sheep or cow with a 1-25 base pair insertion or a 1-25 base pair deletion in a endogenous retroviral provirus gene such as deletion of the sequence for proviral KC. Further specific preferred embodiments include, a cow with a modified milk production gene such as, a cow with a lactase gene insertion in a milk promoter, a cow with the human lactoferrin gene replacing the bovine lactoferrin gene, a monkey with a human therapeutic gene, or a human antibody gene, a cow with the human lipase gene in a milk promoter, a cow with a human gene placed in a transcription initiation site of a milk gene under the control of its endogenous promoter, a cow with a human gene placed in a transcription initiation site of a globin gene under the control of its endogenous globin gene promoter, a cow and goat with a modified urine protein gene, a mammal with a modified endogenous leptin gene, a modified endogenous OTC gene, a modified endogenous CFTR gene or a modified interleukin gene. Additional preferred embodiments include an animal such as a mouse, rabbit or goat with a transcriptional regulation cassette inserted in the transcriptional start site of an integrin gene, and a mouse with a modification in the integrin gene or G-protein coupled receptor gene.

The targeting polynucleotides and recombinase of interest can be transferred into the target cell by well-known methods, depending on the type of cellular host. For example, microinjection, piezo-driven micropipette injection is commonly utilized for target cells, although calcium phosphate treatment, electroporation, lipofection, biolistics or viral-based transfection also may be used (Wolff et al. (1990) Science 247: 1465, Perry et al. Science 284:1180 (1999)) which are incorporated herein by reference). Other methods used to transform mammalian cells include the use of Polybrene, protoplast fusion, and others (see, generally, Sambrook et al. Molecular Cloning: A Laboratory Manual, 2d ed., 1989, Cold Spring Harbor Laboratory Press, Cold Spring Harbor, N.Y., which is incorporated herein by reference).

Generally, any predetermined endogenous DNA sequence, such as a gene sequence, can be altered by enhanced homologous recombination (which includes gene conversion) with an exogenous targeting polynucleotides (such as a complementary pair of single-stranded targeting polynucleotides). The target polynucleotides have at least one homology clamp which substantially corresponds to or is substantially complementary to a predetermined endogenous DNA target sequence and are introduced with a recombinase (e.g., recA) into a target cell having the predetermined endogenous DNA sequence. Typically, a targeting polynucleotide (or complementary polynucleotide pair) has a portion or region having a sequence that is not present in the preselected endogenous targeted sequence(s) (i.e., a nonhomologous portion or mismatch) which may be as small as a single mismatched nucleotide, several mismatches, or may span up to about several kilobases or more of nonhomologous sequence. Generally, such nonhomologous portions are flanked on each side by homology clamps, although a single flanking homology clamp may be used. Nonhomologous portions are used to make insertions, deletions, and/or replacements in a predetermined endogenous targeted DNA sequence, and/or to make single or multiple nucleotide substitutions in a predetermined endogenous target DNA sequence so that the resultant recombined sequence (i.e., a targeted recombinant endogenous sequence) incorporates some or all of the sequence information of the nonhomologous portion of the targeting polynucleotide(s). Thus, the nonhomologous regions are used to make variant sequences, i.e. targeted sequence modifications. Additions and deletions may be as small as 1 nucleotide or may range up to about 2 to 4 kilobases or more. In this way, site directed modifications may be done in a variety of systems for a variety of purposes.

In a preferred application, a targeting polynucleotide is used to repair a mutated sequence of a structural gene by replacing it or converting it to a wild-type sequence (e.g., a sequence encoding a protein with a wild-type biological activity). For example, such applications could be used to convert a sickle cell trait allele of a hemoglobin gene to an allele which encodes a hemoglobin molecule that is not susceptible to sickling, by altering the nucleotide sequence encoding the β-subunit of hemoglobin, so that the codon at position 6 of the β-subunit is converted fromValβ6→Gluβ6 (Shesely et al. (1991) op.cit.). Other genetic diseases can be corrected, either partially or totally, by replacing, inserting, and/or deleting sequence information in a disease allele using appropriately selected exogenous targeting polynucleotides. For example but not for limitation, the ΔF508 deletion in the human CFTR gene can be corrected by targeted homologous recombination employing a recA-coated targeting polynucleotide of the invention.

For the efficient production of transgenic organisms, a target cells must be correctly targeted, with a minimum number of incorrect recombination events. To accomplish this objective, the combination of: (I) a targeting polynucleotide(s), (2) a recombinase (to provide enhanced efficiency and specificity of correct homologous sequence targeting), and (3) a cell-uptake component (to provide enhanced cellular uptake of the targeting polynucleotide), provides a means for the efficient and specific targeting of cells.

Several disease states may be amenable to prophylaxis by targeted alteration of chromosomal sequences in vivo by homologous gene targeting. For example and not by limitation, the following diseases, among others not listed, are expected to be ameliorated by the methods described herein: hepatocellular carcinoma, HBV infection, familial hypercholesterolemia (LDL receptor defect), alcohol sensitivity (alcohol dehydrogenase and/or aldehyde dehydrogenase insufficiency), hepatoblastoma, Wilson's disease, congenital hepatic porphyrias, inherited disorders of hepatic metabolism, ornithine transcarbamylase (OTC) alleles, HPRT alleles associated with Lesch Nyhan syndrome, etc.

In a preferred embodiment, the methods and compositions of the invention are used for gene inactivation. That is, in addition to correcting disease alleles, exogenous targeting polynucleotides can be used to inactivate, decrease or alter the biological activity of one or more genes in a cell (or transgenic nonhuman animal). This finds particular use in the generation of animal models of disease states, or in the elucidation of gene function and activity, similar to “knock out” experiments. These techniques may be used to eliminate a biological function; for example, a galT gene (alpha galactosyl transferase genes) associated with the xenoreactivity of animal tissues in humans may be disrupted to form transgenic animals (e.g. pigs) to serve as organ transplantation sources without associated hyperacute rejection responses. Alternatively, the biological activity of the wild-type gene may be either decreased, or the wild-type activity altered to mimic disease states. This includes genetic manipulation of non-coding gene sequences that affect the transcription of genes, including, promoters, repressors, enhancers and transcriptional activating sequences.

Once the specific target genes to be modified are selected, their sequences may be scanned for possible disruption sites (convenient restriction sites, for example). In one embodiment, plasmids are engineered to contain an appropriately sized gene sequence with a deletion or insertion in the gene of interest and at least one flanking homology clamp which substantially corresponds or is substantially complementary to an endogenous target DNA sequence. Vectors containing a targeting polynucleotide sequence are typically grown in E coli and then isolated using standard molecular biology methods, or may be synthesized as oligonucleotides. Direct targeted inactivation which does not require vectors may also be done. When using microinjection procedures it may be preferable to use a transfection technique with linearized sequences containing only modified target gene sequence and without vector or selectable sequences. The modified gene site is such that a homologous recombinant between the exogenous targeting polynucleotide and the endogenous DNA target sequence can be identified by using carefully chosen primers and PCR, followed by analysis to detect if PCR products specific to the desired targeted event are present (Erlich et al., (1991) Science 252: 1643, which is incorporated herein by reference). Several studies have already used PCR to successfully identify and then clone the desired transfected cell lines (Zimmer and Gruss, (1989) Nature 338: 150; Mouellic et al., (1990) Proc. Natl. Acad. Sci. USA 87: 4712; Shesely et al., (1991) Proc. Natl. Acad. Sci. USA 88: 4294, which are incorporated herein by reference). This approach is very effective (i.e., with microinjection, or with liposomes) and the treated cell populations are allowed to expand to cell groups of approximately 1×10⁴ cells (Capecchi, (1989) Science 244:1288). When the target gene is not on a sex chromosome, or the cells are derived from a female, both alleles of a gene can be targeted by sequential inactivation (Mortensen et al., (1991) Proc. Natl. Acad. Sci. USA 88: 7036).

In addition, the methods of the present invention are useful to add exogeneous DNA sequences, such as exogeneous genes or extra copies of endogeneous genes, to an organism. As for the above techniques, this may be done for a number of reasons, including: to alleviate disease states, for example by adding one or more copies of a wild-type gene or add one or more copies of a therapeutic gene; to create disease models, by adding disease genes such as oncogenes or mutated genes or even just extra copies of a wild-type gene; to add therapeutic genes and proteins, for example by adding tumor suppressor genes such as p53, Rb1, Wt1, NF1, NF2, and APC, or other therapeutic genes; to make superior transgenic animals, for example superior livestock; or to produce gene products such as proteins, for example for protein production, in any number of host cells. Suitable gene products include, but are not limited to, Rad51, alpha-antitrypsin, casein, hormones, antithrombin III, alpha glucosidase, collagen, proteases, viral vaccines, tissue plaminogen activator, monoclonal antibodies, Factors VIII, IX, and X, glutamic acid decarboxylase, hemoglobin, prostaglandin receptor, lactoferrin, calf intestine alkaline phosphatase, CFTR, human protein C, porcine liver esterase, urokinase, and human serum albumin.

Thus, in a preferred embodiment, the targeted sequence modification creates a sequence that has a biological activity or encodes a polypeptide having a biological activity. In a preferred embodiment, the polypeptide is an enzyme with enzymatic activity.

In addition to fixing or creating mutations involved in disease states, a preferred embodiment utilizes the methods of the present invention to create novel genes and gene products. Thus, fully or partially random alterations can be incorporated into genes to form novel genes and gene products, to produce rapidly and efficiently a number of new products which may then be screened, as will be appreciated by those in the art.

In a preferred embodiment, the compositions and methods of the invention are useful in site-directed mutagenesis techniques to create any number of specific or random changes at any number of sites or regions within a target sequence (either nucleic acid or protein sequence), similar to traditional site-directed mutagenesis techniques such as cassette mutagenesis and PCR mutagenesis. Thus, for example, the techniques and compositions of the invention may be used to generate site specific variants at any number of sites. The techniques can be used to make specific changes, or random changes, at a particular site or sites, within a particular region or regions of the sequence, or over the entire sequence.

In this and other embodiments, suitable target sequences include nucleic acid sequences encoding therapeutically or commercially relevant proteins, including, but not limited to, enzymes (proteases, recombinases, lipases, kinases, carbohydrases, isomerases, peptides tautomerases, nucleases etc.), hormones, receptors, transcription factors, growth factors, antibodies, cytokines, globin genes, immunosupppressive genes, tumor suppressors, oncogenes, complement-activating genes, milk proteins (casein, α-lactalbumin, β-lactoglobulin, whey proteins, serum albumin), immunoglobulins, urine proteins, milk proteins, esterases, pharmaceutical proteins and vaccines.

In a preferred embodiment, the methods of the invention are used to generate pools or libraries of variant nucleic acid sequences, and transgenic animal libraries containing the variant libraries. Thus, in this embodiment, a plurality of targeting polynucleotides are used. The targeting polynucleotides each have at least one homology clamp that substantially corresponds to or is substantially complementary to the target sequence. Generally, the targeting polynucleotides are generated in pairs; that is, pairs are made of two single stranded targeting polynucleotides that are substantially complementary to each other (i.e. a Watson strand and a Crick strand). However, as will be appreciated by those in the art, less than a one to one ratio of Watson to Crick strands may be used; for example, an excess of one of the single stranded target polynucleotides (i.e. Watson) may be used. Preferably, sufficient numbers of each of Watson and Crick strands are used to allow the majority of the targeting polynucleotides to form double D-loops, which are preferred over single D-loops, as outlined above. In addition, the pairs need not have perfect complementarity; for example, an excess of one of the single stranded target polynucleotides (i.e. Watson), which may or may not contain mismatches, may be paired to a large number of variant Crick strands, etc. Due to the random nature of the pairing, one or both of any particular pair of single-stranded targeting polynucleotides may not contain any mismatches. However, generally, at least one of the strands will contain at least one mismatch.

The plurality of pairs preferably comprise a pool or library of mismatches. The size of the library will depend on the number of residues to be mutagenized, as will be appreciated by those in the art. Generally, a library in this instance preferably comprises at least 40% different mismatches, with at least 30% mismatches being preferred and at least 10% being particularly preferred. That is, the plurality of pairs comprise a pool of random and preferably degenerate mismatches over some regions or all of the entire targeting sequence. As outlined herein, “mismatches” include substitutions, insertions and deletions. Thus, for example, a pool of degenerate variant targeting polynucleotides covering some, or preferably all, possible mismatches over some region are generated, as outlined above, using techniques well known in the art. Preferably, but not required, the variant targeting polynucleotides each comprise only one or a few mismatches (less than 10), to allow complete multiple randomization, as outlined below.

As will be appreciated by those in the art, the introduction of a pool of variant targeting polynucleotides (in combination with recombinase) to a target sequence can result in a large number of homologous recombination reactions occuring over time. That is, any number of homologous recombination reactions can occur on a single target sequence, to generate a wide variety of single and multiple mismatches within a single target sequence, and a library of such variant target sequences, most of which will contain mismatches and be different from other members of the library. This thus works to generate a library of mismatches.

In a preferred embodiment, the variant targeting polynucleotides are made to a particular region or domain of a sequence (i.e. a nucleotide sequence that encodes a particular protein domain). For example, it may be desirable to generate a library of all possible variants of a binding domain of a protein, without affecting a different biologically functional domain, etc. Thus, the methods of the present invention find particular use in generating a large number of different variants within a particular region of a sequence, similar to cassette mutagenesis but not limited by sequence length. In addition, two or more regions may also be altered simultaneously using these techniques. Suitable domains include, but are not limited to, kinase domains, nucleotide-binding sites, DNA binding sites, signaling domains, structural domains, receptor binding domains, transcriptional activating regions, promoters, origins, active enzyme domains, dimerizing domains, leader sequences, terminators, localization signal domains, and, in immunoglobulin genes, the complementaity determining regions (CDR), Fc, V_{H} and V_{L}.

In a preferred embodiment, the variant targeting polynucleotides are made to the entire target sequence. In this way, a large number of single and multiple mismatches may be made in an entire sequence.

Thus for example, the methods of the invention may be used to create superior recombinant genes such as superior antibiotic and drug resistance genes; superior recombinase genes; and other superior recombinant genes and proteins, including peptides, immunoglobulins, vaccines or other proteins with therapeutic value. For example, targeting polynucleotides containing any number of alterations may be made to one or more functional or structural domains of a protein, and then the products of homologous recombination evaluated.

Once the transgenic organisms are made, the transgenic organism is screened by standard methods, such as Southern, northern, or western blotting, PCR etc. to identify at least one cell that contains the targeted sequence modification. This will be done in any number of ways, and will depend on the target gene and targeting polynucleotides, as will be appreciated by those in the art. The screen may be based on phenotypic, biochemical, genotypic, or other functional changes, depending on the target sequence and the manner in which it is modified. In an additional embodiment, as will be appreciated by those in the art, selectable markers or marker sequences may be included in the targeting polynucleotides to facilitate later identification.

In a preferred embodiment, the gender of the transgenic offspring is sexually skewed, for example, having a disproportionate number of females to males, for example, a ratio that is greater or less than one-to-one. Preferably, the ratio or one gender to the other is at least greater than 50%, more preferably greater than 85%, and most preferably greater than 95% identical. In some embodiments the ratio is 100%.

In a preferred embodiment, the transgenic offspring are infertile and are incapable of sexual reproduction. For example, infertile offspring do not reach sexual maturity or alternatively do not produce functional gametocytes. Such transgenic offspring are maintained by nuclear transfer, intracytoplasmic sperm injection, or other types of in vitro fertilization techniques. In an alternative embodiment, the transgenic offspring are fertile. Fertile transgenic offspring are inbred to produce a population of transgenic organisms or are optionally outbread to introduce the targeted and modified gene of interest into another population of organisms.

In a preferred embodiment, kits containing the compositions of the invention are provided. The kits include the compositions, particularly those of libraries or pools of degenerate cssDNA probes, along with any number of reagents or buffers, including recombinases, buffers, salts, ATP, etc.

The broad scope of this invention is best understood with reference to the following examples, which are not intended to limit the invention in any manner. All patents, patent applications, references, and publications and references cited therein are hereby expressly incorporated by reference in their entirety.

### EXAMPLES

### Example 1

### Transgenic Mice Production with Recombinant Nuclei from Intact Cells

Female B6DF1 mice, 7-11 weeks old are induced to superovulate by i.p. injection of 7.5 IU eCG followed by 7.5 IU hCG. Thirteen hours after hCG injection, cumulus-oocyte complexes are collected from oviducts and treated in HEPES-CZB medium with 0.1% w/v, (300 U/mg) bovine testicular hyaluronidase to disperse the cumulus cells. Cumulus cells of at least about 10-12 micron diameter were selected for EHR modification and nuclear transfer. Dispersed cumulus cells are transferred to HEPES-CZB medium with 10% w/v polyvinylpyrrolidone (average MW, 360,000) and maintained at room temperature for up to 3 hours. (Wakayama et al. Nature. 394:369 (1998).

Nucleoprotein filament probes are prepared by cssDNA probes coated with recombinase protein. A defined series of targeting polynucleotide cssDNA probes designed to target exon 2 of the mouse APRT gene with genetic modifications that range from a single base substitution to the introduction of a 1 kb GFP reporter gene are shown in Table 1. The nucleoprotein filaments are introduced into the cumulus cells by piezo mediated microinjection. Transfected cells are grown for 5 to 14 days and screened for recombinants using PCR and Southern hybridization.

TABLE 1
cssDNA Probes for Targeting the
APRT gene in Adult or Fetal Cells for Nuclear Transfer and
Mammalian Trangenesis by Intracytoplasmic Sperm Injection

| | | | | Size |
|---|---|---|---|---|
| css | | Genetic Modification in | New Sites | cssDNA |
| DNA Probe | Origin | APRT | Inserted | (bp) |
| HAP22I-B | Human | 22 bp insertion | I-Scel | 222 |
| MAP1S-B | Mouse | 1 bp insertion | EcoRV | 220 |
| MAP22I-B | Mouse | 22 bp insertion | I-Scel | 222 |
| MAP22I-E | Mouse | 22 bp insertion | I-Scel | 622 |
| MAP22I-F | Mouse | 22 bp insertion | I-Scel | 1022 |
| MAP_GFP | Mouse | 576 bp insertion of partial | Several | 776 |
| | | GFP gene | | |
| MAPGFP | Mouse | 1009 bp insertion of full- | Several | 1209 |
| | | length GFP | | |

Female B6DF1 mice strain oocytes are obtained 13 hours after hCG injection of eCG-primed females are freed from the cumulus oophorous and maintained in CZB medium, 37.5° C. under 5% (v/v) carbon dioxide until required. Oocytes are transferred into a droplet of HEPES-CZB medium with 5 microgram/ml cytochalasin B. Oocytes are held with a holding pipette and the zone pellucida is cored by several piezo-pulses with an enucleation pipette. Metaphase II chromosome-spindle complexes are aspirated.

Nuclei are removed from the donor cumulus cells and gently aspirated in and out of the injection pipette (˜7 micron inner diameter) until the nuclei were largely devoid of cytoplasm. Each nucleus is injected into a separate enucleated oocyte within 5 minutes of its isolation to form a recombinant zygote (Kimura et al. Development 21:2397-2405 (1995).

The recombinant zygotes are maintained with CZB medium, 37.5° C. under 5% (v/v) carbon dioxide for about 1-6 hours and are activated by the addition of 10 mM Sr²⁺ and 5 microgram/ml cytochalasin B. Recombinant zygotes which divide and develop distinct pseudopronuclei are considered to be activated to form embryos or morulae/blastocysts.

Approximately, 2- to 8-cell embryos or morulae/blastocysts are transferred into oviducts or uteri of surrogate mothers that had been mated with vasectomized males 1 or 3 days previously. Offspring are harvested by caesarean section or allowed to emerge by natural birth and analyzed for the specific transgene modification. (Wakayama et al. Nature 394:369 (1998))

### Example 2

### Transgenic Mice Production with Recombinant Nuclei from Intact Cells

Donor cells are isolated from the tail-tips of adult B6C3F1 male mice, separated from skin, cut into small pieces and incubated in Dulbecco's modified Eagle's medium (DMEM; 5 ml) with 10% feta calf serum (FCS), cultured for about 5-7 days at 37.5° C. under 5% CO₂.

Nucleoprotein filament probes are prepared by recombinase coating the cssDNA probes of Table 1 coated with RecA protein. The nucleoprotein filaments are introduced into the tail-tip cells by microinjection. Transfected cells are growth for 5 to 14 days and screened for recombinants using PCR and Southern hybridization.

The tail-tip cells are trypsinized, washed and placed in a drop of polyvinyl pyrrolidone-supplement CZB medium on a microscope stage (Wakayama et al. supra; Chatot et al. Biol. Reprod. 42:432-440 (1990); Kimura et al. Development 121:2397-2405 (1995)) and separated from the cytoplasm by gentle aspiration. Female mice are induced to superovulate. Oocytes are harvested and maintained as described in Example 1. A single nucleus from a tail-tip cell is injected into an enucleated oocyte, prepared as described in Example 1, to produce a recombinant zygote. The zygote is activated with 10 mM Sr²⁺, 5 micrograms/ml cytochalasin B for 1-3 hours after nuclear transfer to produced embryos of 2-8 cells, morulae, or blastocytes. Following activation,zygotes are transfered to surrogate mothers. Offspring are harvested either by caesarean section or full-term gestation and analyzed. (Wakayama and Yanagimachi. Nature Genetics 22:127 (June 1999)).

### Example 3

### Transgenic Mice Production with Recombinant Nuclei from Permeabilized Cells

Actively growing and growth arrested mouse fibroblast cells are harvested from cultured cells or primary fibroblast cells isolated from fetal mice or adult mouse tails by trypsinization, washing and resuspended in complete DMEM without serum, embedded in 0.5% agarose (Fisher Biotech) in New Buffer (130 mM KCl, 10 mM Na₂HPO₄, 1 mM MgCl₂, 1 mM Na₂-ATP, 1 mM DTT, pH 7.4) and DMEM without serum. The final cell concentration is approximately 8×10⁵ cells/ml to 2.4×10⁷ cells/ml. Embedded cells are permeabilized by the method of Jackson and Cook (1985. A general method for preparing chromatin containing intact DNA. EMBO J. 4:913-918) by treatment with 3 volumes of 0.5% Triton-X-100 in New Buffer at 4° C. for 1 to 10 minutes. Permeabilized cells are incubated with recombinase coated complementary single-stranded nucleoprotein filaments shown in Table 1 for 3 is hours at 37° C. in CF buffer (10 mM Tris-acetate, pH 7.5, 50 mM NaOAc, 2 mM MgOAc, 1 mM DTT), washed 1x in CF buffer and used as donor nuclear for transfer with a piezo impact pipet drive into enucleated oocytres as described in example 1.

### Example 4

### Production of Clonally Derived Rodents by Nuclear Transfer by Microinjection or Piezo-Impact Microinjection

Fibroblasts are harvested from B6D2F1 mice (black coat) and cultured in DMEM supplemented with 10% fetal calf serum in 5% CO₂ for 5-7 days. Nucleoprotein filament probes are prepared by cssDNA probes coated with recombinase protein. A defined series of targeting polynucleotide cssDNA probes designed to target exon 2 of the mouse APRT gene with genetic modifications that range from a single base substitution to the introduction of a 1 kb GFP reporter gene are shown in Table 1. The nucleoprotein filaments are introduced into the fibroblast cells by microinjection, electroporation or chemical transfection. Transfected cells are grown for 5 to 14 days and screened for recombinants using PCR and Southern hybridization.

Female mice strain B6D2F1 and/or B6C3F1 (agout) are induced to ovulate by injection of eCG and hCG. Oocytes are obtained 13 hours after hCG injection of eCG-primed females are freed from the cumulus oophorous and maintained in CZB medium, 37.5° C. under 5% (v/v) carbon dioxide until required. Oocytes are transferred into a droplet of HEPES-CZB medium with 5 microgram/ml cytochalasin B. Oocytes are held with a holding pipette and the zone pellucida is cored by several piezo-pulses to an enucleation pipette. Metaphase II chromosome-spindle complexes are aspirated. Nuclei are removed from the fibroblast cells and gently aspirated in and out of the injection pipette (˜7 micron inner diameter) until the nuclei were largely devoid of cytoplasm. Each nucleus is injected into a separte enucleated oocyte with 5 minutes of its isolation to form a recombinant zygote (Kimura et al. Development 21:2397-2405 (1995).

Recombinant zygote development is activated by incubation in the presence of appropraite concentrations of Sr2+ and cytochalasin B to suppress polar body formation and to allow formation of pseudo-pronuclei. Activated zygotes are cultured to the 2- to 8-cell embryo stage and transplanted into CD-1 albino surrogate mothers. All black B6D2F1 pups are the recombinant offspring from these transferes. All black mice from these experiments are genetically characterized by PCR and Southern DNA hybridization analyses of DNA from tail biopsies. PCR and Southern DNA hybridization analyses are identical those for the parental nucelar donor cell clones.

### Example 5

### Production of Transgenic Mice by Sperm Head Mediated DNA Transfer

B6DF1 female mice, 7-11 weeks old, are induced to superovulate by i.p. injection of 7.5 IU eCG follows by 7.5 IU hCG 48 hours later. Oocytes are collected from oviducts about 16 hours post hCG injection and are prepared and cultured as described (Kimura et al. Biology of Reproduction. 52:709-720 (1995); Kuretake et al. 1996. Biology of Reproduction 55:789-795; Wakayama et al. 1998. Biology of Reproduction 59:100-104).

Spermatozoa are obtained from B6D2F1 male mice (8-12 weeks old). A cauda epididymis is isolated and placed in HEPES-CZB, large tubules are cut to allow spermatozoa to escape. Spermatozoa are collected and treated as described by Wakayama et al. Nature Biotechnol. 16:639 (1998). Spermatozoa are untreated or are subjected to either freeze-thawing (Wakayam et al. J. Fertil.

Reprod. 112, 11:(1998)); freeze-drying (Wakayama et al. Nature Biotechnol. 16:639 (1998)); or Triton-X-100 extract (Perry et al. Science 284:1180 (1999)). The treated and untreated spermatozoa are 25 mixed with nucleoprotein filaments prepared as described in Example 1 in CZB or NIM media and incubated at 25° C. or on ice for 1 minutes.

Nucleoprotein filament-spermatozoa complexes are mixed with a polyvinylpyrrolidone (PVP, average MW-360,000) solution to give a final concentration of about 10% (w/v) PVP. Injections are performed with a piezo-actuated microinjection in CZB−=H at room temeprature within 1 hour of spermatozoa-nucleofilament mixing or within 1 hour of spermatozoa-Triton-X-100 mixing. About 1 picoliter of nucleofiling/spermatozoa mixture is microinjected into the oocyte. For microinjection, spermatozoa are aspirated into a pipette attached to a piezoelectric pipette-driving unit and on spermatozoa is injected per oocyte as described in Kimura et al. Biol. Reprod. 52:709 (1995) and Huang et al. 1996. Journal of Assisted Reproduction and Genetics. 13:320-328 to produce a recombinant zygotes. Dislocation of heads from tails is done by the applicationof a single piezo pulse as described. Recombinant zygotes are treated with 10 mM SrCl2 and 5 micrograms/ml cytocholasin B, incubated under standard embryo culture conditions and transfered to surrogate mothers prepared as previously described (Wakayama et al. 1998. Nature 394:369-373; Wakayama et al. 1999. Nature Genetics. 22:127-128; Perry et al. 1999. Science 284:1180-1183; Kimura et al. 1995. Biology of Reproduction 52:709-720.

We claim:

1. A method comprising:
a) altering a chromosomal sequence of a donor nucleus of a donor cell by introducing a pair of single-stranded targeting polynucleotides, and a recombinase into said donor nucleus of said donor cell, wherein said pair of targeting polynucleotides are substantially complementary to each other and each comprising a homology clamp that substantially corresponds to or is substantially complementary to a predetermined DNA sequence of said nucleus; and,
b) transplanting said nucleus into an oocyte to produce a recombinant zygote.

2. The method of claim 1 further comprising:
c) activating said recombinant zygote.

3. The method of claim 1 or 2 further comprising:
d) transfering said recombinant zygote into a surrogate mother.

4. The method of claim 3 further comprising:
e) harvesting a transgenic offspring of said mother.

5. The method of claim 4 further comprising:
f) breeding said offspring.

6. The method of claim 1 wherein said recombinase is RecA.

7. The method of claim 6 wherein said RecA is E. coli RecA.

8. The method of claim 1 wherein said recombinase is Rad51.

9. The method of claim 1 wherein said donor nucleus is an isolated nucleus.

10. The method of claim 1 wherein said donor cell is selected from the group consisting of a haploid cell, a diploid cell, a somatic cell, an embryonal cell, and a fetal cell .

11. The method of claim 10 wherein said haploid cell is selected from the group consisting of a germ cell, a germ cell precursor, a germ stem cell, and a gametocyte.

12. The method of claim 10 wherein said somatic cell is selected from the group consisting of a mammary derived cell, an adult tail-tip cell, a cumulus cell, an epithelial cell, a dermal cell, a keratinocyte, a mesenchymal cell, a stem cell, a blood cell, and a fibroblast.

13. The method of claim 10 wherein said embryonal cell is selected from the group consisting of an embryonal germ cell, an embryonal stem cell, an umbilical cord cell, an umbilical cord blood cell, an endodermal cell, a mesodermal cell, and an endodermal cell.

14. The method of claim 1 wherein said oocyte is an enucleated oocyte.

15. The method of claim 1 wherein said oocyte is arrested in metaphase of meiosis II.

16. The method of claim 1 wherein said oocyte is selected from the group consisting of a rodent, ungulate, bovine, ovine, canine, feline, simian, rabbit, equine, fish, amphibian, reptile, crustacean, and mollusc oocyte.

17. The method of claim 1 wherein said transplanting is by microinjection, electrofusion, or piezo driven micropipet injection.

18. The method of claim 2 wherein said activating occurs about 6 hours or less after said transferring step.

19. The method of claim 2 wherein said activating is by electroactivation.

20. The method of claim 2 wherein said activating is by contacting said recombinant zygote with a chemical activator.

21. The method of claim 20 wherein said activator is selected from the group consisting of Ca²⁺ release stimulators, Ca²⁺ ionophores, strontium ions, sperm cytoplasmic factors, inhibitors of protein synthesis, oocyte receptor ligand mimetics, regulators of phosphoprotein signaling, and ethyl alcohol.

22. A method comprising introducing a spermatozoa, a pair of single-stranded targeting polynucleotides, and a recombinase into an oocyte, wherein said pair of targeting polynucleotides are substantially complementary to each other and each comprising a homology clamp that substantially corresponds to or is substantially complementary to a predetermined DNA sequence of said spermatozoa and/or said oocyte whereby a recombinant zygote is produced.

23. The method of claim 22 further comprising:
b) activating said recombinant zygote.

24. The method of claim 23 further comprising:
c) transferring said recombinant zygote into a surrogate mother.

25. The method of claim 24 further comprising:
d) harvesting the transgenic offspring of said mother.

26. The method of claim 25 further comprising:
e) breeding said offspring.

27. The method of claim 22 wherein said recombinase is RecA.

28. The method of claim 27 wherein said RecA is E. coli RecA.

29. The method of claim 22 wherein said recombinase is Rad51.

30. The method of claim 22 wherein said spermatozoa is a sperm head.

31. A composition comprising a spermatozoa and at least one nucleoprotein filament.

32. The composition of claim 31 wherein said spermatozoa is a sperm head.

33. The composition of claim 31 wherein said sperm head is a freeze-dried and rehydrated sperm head.

34. The composition of claim 31 wherein said sperm head is a demembranated sperm head.

35. The composition of claim 31 wherein said sperm head is a detergent-treated sperm head.

36. The composition of claim 30 wherein said at least one nucleoprotein filament comprises at least one homologous motif tag sequence.

37. The composition of claim 36 comprising a second nucleoprotein filament comprising a second homologous motif tag sequence.

38. A method of altering a nucleic acid sequence of a mitochondria or chloroplast of a cell comprising: introducing into a cell a pair of single-stranded targeting polynucleotides, and a recombinase, wherein said pair of targeting polynucleotides are substantially complementary to each other, and each comprising a homology clamp that substantially corresponds to or is substantially complementary to a predetermined nucleic acid sequence of said mitochondria or chloroplast, whereby said sequence is altered.

39. The method of claim 38 wherein said cell is a plant cell.

40. The method of claim 38 wherein said pair of single-stranded targeting polynucleotides, and a recombinase are introduced into said cell by biolistics.
